(19) United States
(12) Patent Application Publication
(10) Pub. No.: US 2008/0318878 A1
(43) Pub. Date: Dec. 25, 2008

(54) Antibacterial Agents

(76) Inventors: Biswajit Das, Haryana (IN); Rita Katoch, Haryana (IN); Atul Kashinath Hajare, Maharashtra (IN); Anish Bandyopadhyay, Haryana (IN); Santosh Haribhau Kurhade, Maharashtra (IN); Sujata Rathy, Delhi (IN); Ashok Rattan, Delhi (IN)

Correspondence Address: Jayadeep R. Deshmukh, Esq.;Ranbaxy Inc., 600 College Road East, Suite 2100, Princeton, New Jersey, NJ, 08540, US

(21) Appl. No.: 11/576,033

(22) PCT Filed: Sep. 27, 2005
(86) PCT No.: PCT/IB05/02894

§ 371 Date: Jun. 20, 2008

(30) Foreign Application Priority Data
Sep. 27, 2004 (IN) 1851/DEL/2004

(51) Int. Cl. A61K 31/7048; C07H 17/08

(52) U.S. Cl. 514/29; 536/7.4

(57) ABSTRACT

The present invention provides acylide derivatives, which can be used as antibacterial agents. Compounds disclosed herein can be used for treating or preventing conditions caused by or contributed to by Gram-positive, Gram-negative or anaerobic bacteria, more particularly against, for example, Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus, Enterobactericeae or any combination thereof. Also provided are processes for preparing compounds disclosed herein, pharmaceutical compositions thereof, and method of treating bacterial infections.

### FIELD OF THE INVENTION

The present invention provides acylide derivatives, which can be used as antibacterial agents. Compounds disclosed herein can be used for treating or preventing conditions caused by or contributed to by Gram-positive, Gram-negative or anaerobic bacteria, more particularly against, for example, Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus, Enterobactericeae or any combination thereof. Also provided are processes for preparing compounds disclosed herein, pharmaceutical compositions thereof, and method of treating bacterial infections.

### BACKGROUND OF THE INVENTION

First generation macrolides erythromycin A and the early derivatives are characterized by bacteriostatic or bactericidal activity for most Gram-positive bacteria, atypical pathogens, and many community acquired respiratory infections and in patients with penicillin allergy. However, erythromycin A causes numerous drug-drug interactions, has relatively poor absorption, poor local tolerance, loses its antibacterial activity under acidic conditions by degradation and the degraded products are known to be responsible for undesired side effects. (Itoh, Z et al., Am. J. Physiol., 1984, 247:688; Omura, S et al., J. Med. Chem., 1987, 30:1943). Various erythromycin A derivatives have been prepared to overcome the acid instability and other problems associated with it.

Roxithromycin, clarithromycin and azithromycin were developed to address the limitation of erythromycin A. Both clarithromycin and azithromycin were found to be important drugs in the treatment and prophylaxis of atypical mycobacterial infections in patients with HIV.

Macrolides were found to be effective drugs in the treatment of many respiratory tract infections. However, increasing resistance among S. pneumoniae has prompted the search for new compounds that retain the favorable safety profile, and a spectrum of activity and are confined to respiratory pathogens. Consequently, numerous investigators have prepared chemical derivatives of erythromycin A in an attempt to obtain analogs having modified or improved profiles of antibiotic activity.

PCT Publication No. WO 99/11651 discloses 3-descladinosyl-6-O-substituted erythromycin derivatives, which have been said to be useful as antibacterial agents. PCT Publication No. WO 02/12260 discloses macrolide antibiotics. PCT Publication Nos. WO 01/10878; 01/10879 and 01/10880 disclose novel erythromycin derivatives stated to have potent antibacterial effects on erythromycin-resistant bacteria and Haemophilus influenzae. U.S. Pat. No. 6,140,479 discloses erythromycin derivatives stated to have antibacterial activity.

### SUMMARY OF THE INVENTION

The present invention provides acylide derivatives, which can be used in the treatment or prevention of bacterial infection, and processes for the synthesis of these compounds.

Pharmaceutically acceptable salts, pharmaceutically acceptable solvates, stereoisomers, tautomers, racemates, prodrugs, metabolites, polymorphs of these compounds having same type of activity are also provided.

Pharmaceutical compositions containing the disclosed compounds together with pharmaceutically acceptable carriers, excipients or diluents, which can be used for the treatment of bacterial infection.

Thus in one aspect, provided herein are compounds having the structure of Formula I,

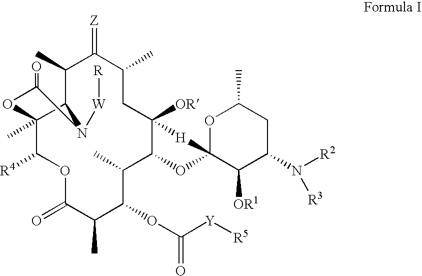

pharmaceutically acceptable salts, pharmaceutically acceptable solvates, stereoisomers, tautomers, racemates, prodrugs, metabolites and polymorphs thereof, wherein:
R¹ can be hydrogen or a hydroxyl protecting group;
R² and R³ can independently be hydrogen, alkyl, alkenyl, alkynyl, cycloalkyl, aryl, heterocycle, aralkyl or (heterocycle)alkyl, with the proviso that R² and R³ are not simultaneously methyl;
R⁴ can be hydrogen, alkyl, alkenyl or alkynyl;
R⁵ can be aryl, heterocycle or alkyl;
R can be no atom, aryl or heterocycle;
R′ can be alkyl or —(CH₂)_{q}—U—V, wherein
   q can be an integer of from 1 to 4;
   U can be alkenyl or alkynyl; and
   V can be hydrogen, aryl or heterocycle;
W can be hydrogen or —(CH₂)ₘ— wherein
   m can be an integer of from 2 to 6;
   CH₂ of (CH₂)ₘ group can be optionally interrupted by O, S or NR⁶, wherein R⁶ can be hydrogen, alkyl, cycloalkyl, alkenyl, heterocyclyl, (heterocyclyl)alkyl, alkynyl, aryl or aralkyl;
   one of the hydrogen atom of (CH₂)ₘ can be optionally replaced by alkyl, hydroxy or alkoxy;
Y can be -Q(CH₂)ₖ—, wherein
   k can be an integer of from 1 to 6;
   Q can be no atom, O or NR⁷, wherein R⁷ can be hydrogen or alkyl;
   one of the hydrogen atom of (CH₂)ₖ can be optionally replaced by alkyl, hydroxy or alkoxy; and
Z can be O, S or NOR⁸, wherein R⁸ can be hydrogen, alkyl or aralkyl.

These compounds can include one or more of the following embodiments. For example, W can be —(CH₂)ₘ—; R can be heterocycle substituted with —NHCONHR⁹, wherein R⁹ can be alkyl, alkenyl, alkynyl, cycloalkyl, aryl, aralkyl, (heterocycle)alkyl or heterocyclyl and m can be an integer of from 2 to 6. In another embodiment, W can be hydrogen or —(CH₂)ₘ—, wherein (CH₂)ₘ group can be optionally interrupted by O, S or NR⁶, wherein R⁶ can be hydrogen, alkyl, cycloalkyl, alkenyl, heterocyclyl, (heterocyclyl)alkyl, alkynyl, aryl or aralkyl; and R can be no atom, aryl or heterocyclyl. In another embodiment, W can be hydrogen or —(CH₂)ₘ—, wherein one of the hydrogen atom of (CH₂)ₘ can be optionally replaced by alkyl, hydroxy or alkoxy. In another embodiment, R² can be methyl, and R³ can be alkyl or alkenyl, with the proviso that R³ is not methyl. In another embodiment, R⁵ can be monocyclic heterocycle having N as heteroatom(s). In a particular embodiment, R can be selected from benziimidazol-1-yl, (1H-imidazo[4,5-b]pyridin-1-yl, (3H-imidazo[4,5-b]pyridin-3-yl, (4-pyridin-3-yl)-phenyl, 1-butyl-3-(9H-purin-6-yl) urea, 1-(2,6-difluoro-phenyl)-3-(9H-purin-6-yl) urea, 1-allyl-3-(9H-purin-6-yl)-urea, 1-(4-fluoro-phenyl)-3-(9H-purin-6-yl urea, (3-pyridin-3-yl)-phenyl and (3-thiophen-3-yl)-phenyl.

In another aspect, provided herein are compounds selected from:
11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-(benzoimidazol-1-yl)-pentyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(benzoimidazol-1-yl)-pentyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(1H-imidazo[4,5-b]pyridin-1-yl)-pentyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(3H-imidazo[4,5-b]pyridin-3-yl)-pentyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-pyridin-3-yl-phenoxy)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((1-(9-(4-amino-butyl)-9H-purin-6-yl)-3-butyl)-urea)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((1-(9-(4-amino-butyl)-9H-purin-6-yl)-3-(2,6-difluoro-phenyl)-urea)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((1-allyl-3-(9-(4-amino-butyl)-9H-purin-6-yl)-urea)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((1-(9-(4-amino-butyl)-9H-purin-6-yl)-3-(4-fluoro-phenyl)-urea)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(3-pyridin-3-yl-phenoxy)-propyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(3-thiophen-3-yl-phenoxy)-propyl)-imino)]erythromycin A,

or pharmaceutically acceptable salts, pharmaceutically acceptable solvates, stereoisomers, tautomers, racemates, prodrugs, metabolites or polymorphs thereof.

In yet another aspect, provided herein are pharmaceutical compositions comprising therapeutically effective amounts of one or more compounds disclosed herein together with one or more pharmaceutically acceptable carriers, excipients, or diluents.

In another aspect, provided herein are methods for treating or preventing conditions caused by or contributed to by Gram-positive, Gram-negative or anaerobic bacteria comprising administering to a mammal in need thereof therapeutically effective amounts of one or more compounds or pharmaceutical compositions disclosed herein.

The methods may include one or more of the following embodiments. For example, the conditions can be selected from community acquired pneumonia, upper and lower respiratory tract infections, skin and soft tissue infections, hospital acquired lung infections or bone and joint infections, mastitis, catether infection, foreign body, prosthesis infections or peptic ulcer disease. In another embodiment, the bacterial infection can be caused by Gram-positive, Gram-negative or anaerobic bacteria.

In yet another embodiment, the Gram-positive, Gram-negative or anaerobic bacteria can be selected from Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus or Enterobactericeae. In a particular embodiment, the bacterium is cocci. In another particular embodiment, the cocci is drug resistant.

In another aspect, provided herein are processes for preparing compounds of Formula XII.

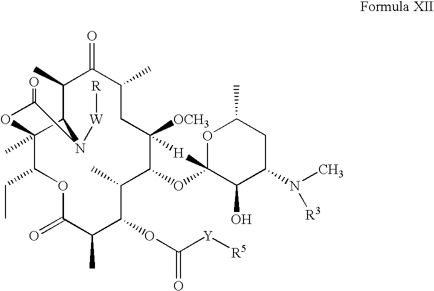

or their pharmaceutically acceptable salts, pharmaceutically acceptable solvates, stereoisomers, tautomers, racemates, prodrugs, metabolites or polymorphs, wherein:

R³ can be hydrogen, alkyl alkenyl, alkynyl, cycloalkyl, aryl, heterocycle, aralkyl or (heterocycle)alkyl, with the proviso that R³ is not methyl;

R⁵ can be aryl, heterocycle or alkyl;

R can be no atom, aryl or heterocycle;

W can be hydrogen or —(CH₂)ₘ—, wherein
m can be an integer of from 2 to 6;
(CH₂)ₘ group can be optionally interrupted by O, S or NR⁶, wherein R⁶ can be hydrogen, alkyl, cycloalkyl, alkenyl, heterocyclyl, (heterocyclyl)alkyl, alkynyl, aryl or aralkyl;
one of the hydrogen atom of (CH₂)ₘ can be optionally replaced by alkyl, hydroxy or alkoxy;
Y can be -Q(CH₂)ₖ—, wherein
   k can be an integer of from 1 to 6;
   Q can be no atom, O or NR⁷, wherein R⁷ is hydrogen or alkyl;
one of the hydrogen atom of (CH₂)ₖ is optionally replaced by alkyl, hydroxy or alkoxy;
the method comprising:
(a) hydrolyzing clarithromycin of Formula II,

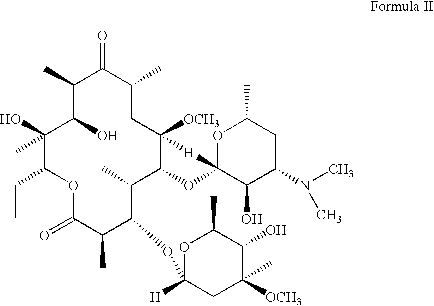

to form a compound of Formula III,

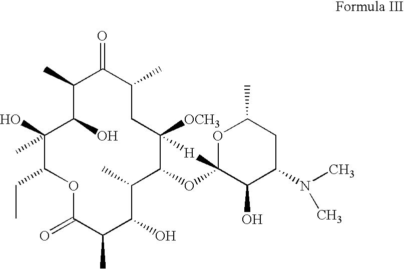

(b) protecting the compound of Formula III by reacting one or more reagents of Formula R¹₂O or R¹X (wherein X can be halogen) to form a compound of Formula IV,

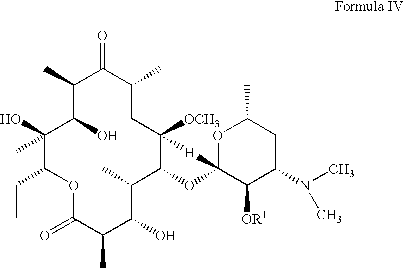

(c) reacting the compound of Formula IV with one or more reagents to form a compound of Formula V,

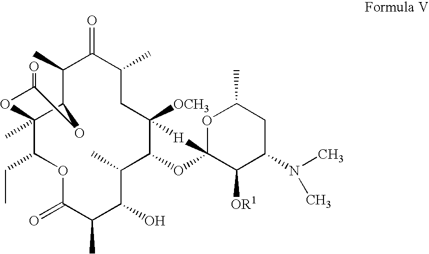

(d) reacting the compound of Formula V with one or more organic bases to form a compound of Formula VI,

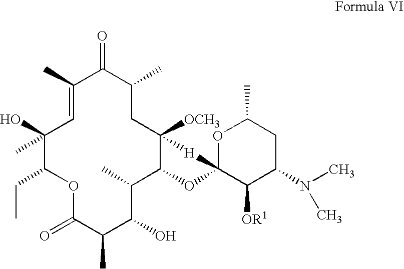

(e) desmethylating the compound of Formula VI at 3′-N-dimethyl group to form a compound of Formula VII,

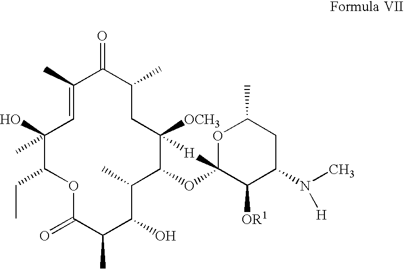

(f) alkylating the compound of Formula VII with one or more reagents of Formula R³CHO, R³₂CO or R³X to form a compound of Formula VIII (wherein R³ can be the same as defined earlier),

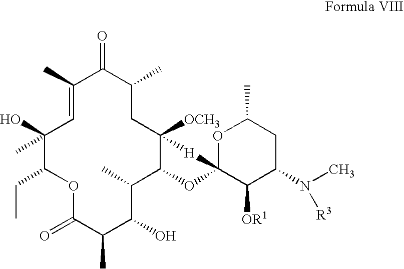

(g) acylating the compound of Formula VIII with one or more reagents of Formula R⁵YCOOH, (R⁵YCO)₂O, R⁵YCOX or R⁵YCOOR¹⁰ (wherein R¹⁰ can be a leaving group, for example, pivaloyl, p-toleuensulfonyl, isobutoxycarbonyl, ethoxycarbonyl or isopropoxycarbonyl) to form a compound of Formula IX (wherein Y and R⁵ can be the same as defined earlier),

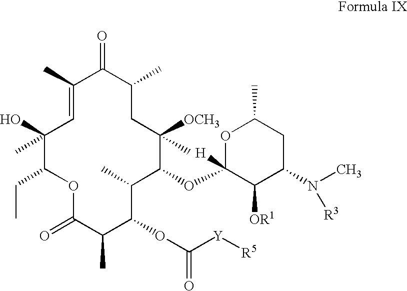

(h) reacting the compound of Formula IX with N,N′-carbonyl diimidazole to form a compound of Formula X,

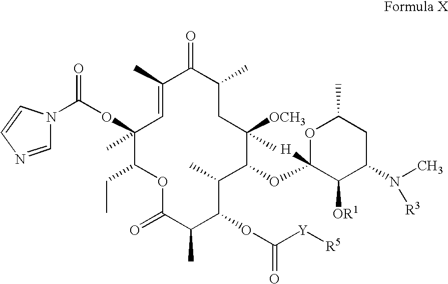

(i) reacting the compound of Formula X with a compound of Formula R—W—NH₂ to form a compound of Formula XI (wherein R and W can be the same as defined earlier),

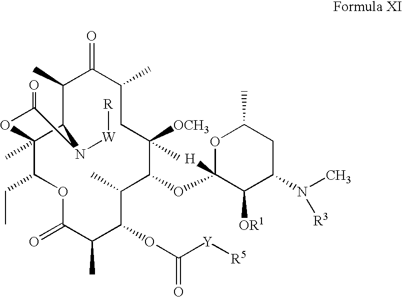

(b) deprotection of the compound of Formula XI to form a compound of Formula XII.
In another aspect, provided herein are processes for preparing compounds of Formula XV,

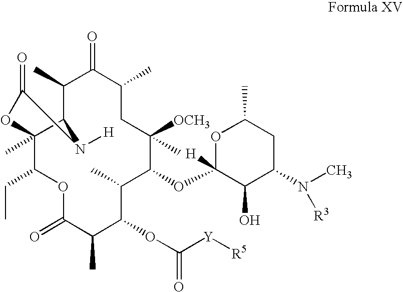

or their pharmaceutically acceptable salts, pharmaceutically acceptable solvates, stereoisomers, tautomers, racemates, prodrugs, metabolites or polymorphs thereof, wherein:
R³ can be hydrogen, alkyl, alkenyl, alkynyl, cycloalkyl, aryl, heterocycle, aralkyl or (heterocycle)alkyl, with the proviso that R³ is not methyl;
R⁵ can be aryl, heterocycle or alkyl;

Y can be -Q(CH₂)ₖ—, wherein
k can be an integer of from 1 to 6;
Q can be no atom, O or NR⁷, wherein R⁷ can be hydrogen or alkyl;
one of the hydrogen atom of (CH₂)ₖ can be optionally replaced by alkyl, hydroxy or alkoxy;

the method comprising:
(a) reacting the compound of Formula X,

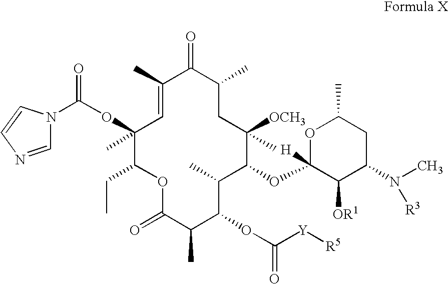

with ammonia to form a compound of Formula XIII (wherein R³, R¹, Y and R⁵ can be the same as defined earlier),

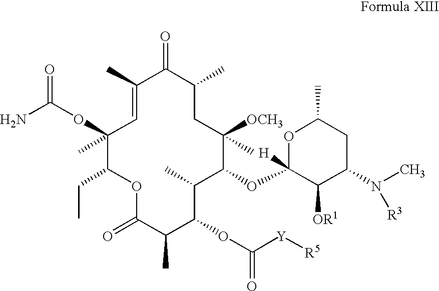

(b) cyclizing the compound of Formula XIII to form a compound of Formula XIV,

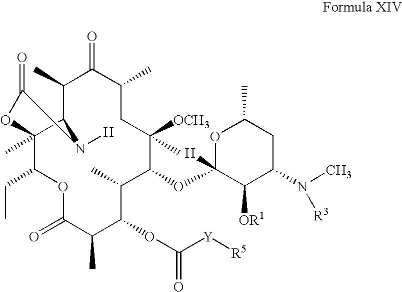

(c) deprotecting the compound of Formula XIV to form a compound of Formula XV.

In another aspect, provided herein is a process for preparing compounds of Formula XIX,

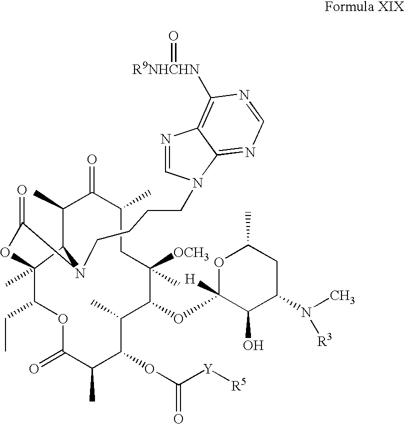

or their pharmaceutically acceptable salts, pharmaceutically acceptable solvates, stereoisomers, tautomers, racemates, prodrugs, metabolites or polymorphs thereof, wherein:
R³ can be hydrogen, alkyl, alkenyl, alkynyl, cycloalkyl, aryl, heterocycle, aralkyl or (heterocycle)alkyl, with the provisio that R³ is not methyl;

R⁵ can be aryl, heterocycle or alkyl;

Y can be -Q(CH₂)ₖ—, wherein
k can be an integer of from 1 to 6;
Q can be no atom, O, NR⁷, wherein R⁷ can be hydrogen or alkyl;
one of the hydrogen atom of (CH₂)ₖ can be optionally replaced by alkyl, hydroxy or alkoxy;
R⁹ can be alkyl, alkenyl, alkynyl, cycloalkyl, aryl, aralkyl, (heterocycle)alkyl or heterocyclyl;

which method comprises the steps of:
(a) reacting the compound of Formula XVI with a compound of Formula XVII,

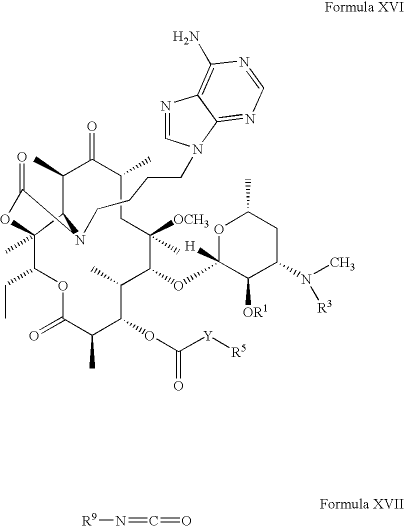

to form a compound of Formula XVIII (wherein R¹, R⁹, R³, Y and R⁵ can be the same as defined earlier),

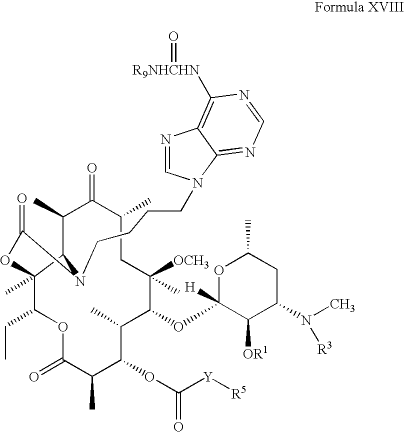

(b) deprotecting the compound of Formula XVIII to form a compound of Formula XIX.

### DETAILED DESCRIPTION OF THE INVENTION

In accordance with one aspect, provided herein are compounds having the structure of Formula I,

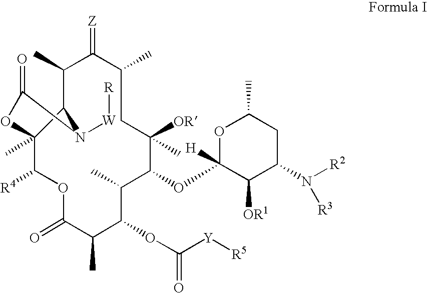

pharmaceutically acceptable salts, pharmaceutically acceptable solvates, stereoisomers, tautomers, racemates, prodrugs, metabolites and polymorphs thereof, wherein:
R¹ can be hydrogen or a hydroxyl protecting group;
R² and R³ can independently be hydrogen, alkyl, alkenyl, alkynyl, cycloalkyl, aryl, heterocycle, aralkyl or (heterocycle)alkyl, with the proviso that R² and R³ are not simultaneously methyl;
R⁴ can be hydrogen, alkyl, alkenyl or alkynyl;
R⁵ can be aryl, heterocycle or alkyl;
R can be no atom, aryl or heterocycle;
R′ can be alkyl or —(CH₂)_{q}—U—V, wherein
   q can be an integer of from 1 to 4;
   U can be alkenyl or alkynyl; and
   V can be hydrogen, aryl or heterocycle;
W can be hydrogen or —(CH₂)ₘ— wherein
   m can be an integer of from 2 to 6;
   CH₂ of (CH₂)ₘ group can be optionally interrupted by O, S or NR⁶, wherein R⁶ can be hydrogen, alkyl, cycloalkyl, alkenyl, heterocyclyl, (heterocyclyl)alkyl, alkynyl, aryl or aralkyl;
   one of the hydrogen atom of (CH₂)ₘ can be optionally replaced by alkyl, hydroxy or alkoxy;
Y can be -Q(CH₂)ₖ—, wherein
   k can be an integer of from 1 to 6;
   Q can be no atom, O or NR⁷, wherein R⁷ can be hydrogen or alkyl;
   one of the hydrogen atom of (CH₂)ₖ can be optionally replaced by alkyl, hydroxy or alkoxy; and
Z can be O, S or NOR⁸, wherein R⁸ can be hydrogen, alkyl or aralkyl.

In one embodiment, W can be —(CH₂)ₘ—; R can be heterocycle substituted with —NHCONHR⁹, wherein R⁹ can be alkyl, alkenyl, alkynyl, cycloalkyl, aryl, aralkyl, heterocycle)alkyl or heterocyclyl and m can be an integer of from 2 to 6. In another embodiment, W can be hydrogen or —(CH₂)ₘ—, wherein (CH₂)ₘ group can be optionally interrupted by O, S or NR⁶, wherein R⁶ can be hydrogen, alkyl, cycloalkyl, alkenyl, heterocyclyl, (heterocyclyl)alkyl, alkynyl, aryl or aralkyl; and R can be no atom, aryl or heterocyclyl. In yet another embodiment, W can be hydrogen or —(CH₂)ₘ, wherein one of the hydrogen atom of (CH₂)ₘ can be optionally replaced by alkyl, hydroxy or alkoxy.

In accordance with a further aspect, provided herein are methods for treating or preventing a mammal suffering from conditions caused by or contributed to by Gram-positive, Gram-negative or anaerobic bacteria comprising administering to a mammal therapeutically effective amounts of one or more compounds or one or more pharmaceutical compositions disclosed herein. Bacterial infection may be caused by one or more bacteria, for example, Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus or Enterobactericeae. The conditions treated or prevented may be, for example, community acquired pneumonia, upper and lower respiratory tract infections, skin and soft tissue infections, hospital acquired lung infections or bone and joint infections, and other bacterial infections such as mastitis, catether infection, foreign body, prosthesis infections or peptic ulcer disease.

In accordance with another aspect, provided herein are processes for preparing compounds disclosed herein.

The term “alkyl,” unless otherwise specified, refers to a monoradical branched or unbranched saturated hydrocarbon chain having from 1 to 20 carbon atoms. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, aryl, heterocyclyl, heteroaryl, arylthio, thiol, alkylthio, aryloxy, nitro, aminosulfonyl, aminocarbonylamino, —NHC(═O)R_{f}, —NR_{f}R_{q}, —C(═O)NR_{f}R_{q}, —NHC(═O)NR_{f}R_{q}, —C(═O)heteroaryl, (═O)heterocyclyl, O—C(═O)NR_{f}R_{q} {wherein R_{f} and R_{q} are independently selected from alkyl, alkenyl, cycloalkyl, cycloalkenyl, aryl, aralkyl, heterocyclyl, heteroaryl, heterocyclylalkyl, heteroarylalkyl}, nitro, or —SO₂R₆ (wherein R₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, carboxy, —NR_{f}R_{q}, —C(═O)NR_{f}R_{q}, —OC(═O)NR_{f}R_{q}, —NHC(═O)NR_{f}R_{q} (wherein R_{f} and R_{q} are the same as defined earlier), hydroxy, alkoxy, halogen, CF₃, cyano, and —SO₂R₆, (wherein R₆ are the same as defined earlier); or an alkyl group also may be interrupted by 1-5 atoms of groups independently selected from oxygen, sulfur or —NRₐ— {wherein Rₐ is selected from hydrogen, alkyl, cycloalkyl, alkenyl, cycloalkenyl, alkynyl, aryl, acyl, aralkyl, —C(═O)OR_{f} (wherein R_{f} is the same as defined earlier), SO₂R₆ (where R₆ is as defined earlier), or —C(═O)NR_{f}R_{q} (wherein R_{f} and R_{q} are as defined earlier)}. Unless otherwise constrained by the definition, all substituents may be substituted further by 1-3 substituents selected from alkyl, carboxy, —NR_{f}R_{q}, —C(═O)NR_{f}R_{q}, —O—C(═O)NR_{f}R_{q} (wherein R_{f} and R_{q} are the same as defined earlier) hydroxy, alkoxy, halogen, CF₃, cyano, and —SO₂R₆ (where R₆ is same as defined earlier); or an alkyl group as defined above that has both substituents as defined above and is also interrupted by 1-5 atoms or groups as defined above.

The term “alkylene,” as used herein, refers to a diradical branched or unbranched saturated hydrocarbon chain having from 1 to 6 carbon atoms and one or more hydrogen can optionally be substituted with alkyl, hydroxy, halogen or oximes. This term can be exemplified by groups such as methylene, ethylene, propylene isomers (e.g, —CH₂CH₂CH₂ and —CH(CH₃)CH₂) and the like. Alkylene may further be substituted with one or more substituents such as alkyl, alkenyl, alkynyl, alkoxy, cycloalkyl, acyl, acylamino, acyloxy, alkoxycarbonylamino, azido, cyano, halogen, hydroxy, oxo, thiocarbonyl, carboxy, arylthio, thiol, alkylthio, aryloxy, heteroaryloxy, aminosulfonyl, —COOR₂ (wherein R₂ is the same as defined earlier), —NHC(═O)Rₓ, —NRₓR_{y}, —C(═O)NRₓR_{y}, —NHC(═O)NRₓR_{y}, —C(═O)heteroaryl, C(═O)heterocyclyl, —O—C(═O)NRₓR_{y} (wherein Rₓ and R_{y} are the same as defined earlier), nitro, —S(O)ₙR₃ (wherein n and R₃ are the same as defined earlier). Unless otherwise constrained by the definition, all substituents may be further substituted by 1-3 substituents chosen from alkyl, carboxy, —COOR₂ (wherein R₂ is the same as defined earlier), —NRₓR_{y}, —C(═O)NRₓR_{y}, —OC(═O)NRₓR_{y}, —NHC(═O)NRₓR_{y} (wherein Rₓ and R_{y} are the same as defined earlier), hydroxy, alkoxy, halogen, CF₃, cyano, and —S(O)ₙR₃ (where R₃ and n are the same as defined earlier). Alkylene can also be optionally interrupted by 1-5 atoms of groups independently chosen from oxygen, sulfur and —NRₐ, where Rₐ is chosen from hydrogen, alkyl, cycloalkyl, alkenyl, alkynyl, aryl, acyl, aralkyl, —C(═O)OR₂ (wherein R₂ is the same as defined earlier), —S(O)ₙR₃ (where n and R₃ are the same as defined earlier), —C(═O)NRₓR_{y} (wherein Rₓ and R_{y} are as defined earlier)-CONH—, —C═O or —C═NOH.

The term “alkenyl,” unless otherwise specified, refers to a monoradical of a branched or unbranched unsaturated hydrocarbon group having from 2 to 20 carbon atoms with cis, trans, or geminal geometry. In the event that alkenyl is attached to a heteroatom, the double bond cannot be alpha to the heteroatom. Alkenyl groups may be substituted further with one or more substituents selected from alkyl, alkynyl, alkoxy, cycloalkyl, cycloalkenyl, acyl, acylamino, acyloxy, —NHC(═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), alkoxycarbonylamino, azido, cyano, halogen, hydroxy, oxo, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, nitro, or SO₂R₆ (wherein R₆ are is same as defined earlier). Unless otherwise constrained by the definition, alkenyl substituents optionally may be substituted further by 1-3 substituents selected from alkyl, carboxy, hydroxy, alkoxy, halogen, —CF₃, cyano, —NR_{f}R_{q}, —C(═O)NR_{f}R_{q}, —O—C(═O)NR_{f}R_{q} (wherein R_{f} and R_{q} are the same as defined earlier) and —SO₂R₆(where R₆ is same as defined earlier).

The term “alkenylene” unless otherwise specified, refers to a diradical of a branched or unbranched unsaturated hydrocarbon group preferably having from 2 to 6 carbon atoms with cis, trans or geminal geometry. In the event that alkenylene is attached to the heteroatom, the double bond cannot be alpha to the heteroatom. The alkenylene group can be connected by two bonds to the rest of the structure of compound of Formula I. Alkenylene may further be substituted with one or more substituents such as alkyl, alkynyl, alkoxy, cycloalkyl, acyl, acylamino, acyloxy, —NHC(═O)Rₓ, —NRₓ, R_{y}, —C(═O)NRₓR_{y}, —NHC(═O)NRₓR_{y}, —OC(═O)NRₓR_{y} (wherein Rₓ, and R_{y} are the same as defined earlier), alkoxycarbonylamino, azido, cyano, halogen, hydroxy, oxo, thiocarbonyl, carboxy, —COOR₂ (wherein R₂ is the same as defined earlier), arylthio, thiol, alkylthio, aryl, aralkyl, aryloxy, heterocyclyl, heteroaryl, heterocyclyl alkyl, heteroaryl alkyl, aminosulfonyl, alkoxyamino, nitro, —S(O)ₙR₃ (where R₃ and n are the same as defined earlier). Unless otherwise constrained by the definition, all substituents may optionally be further substituted by 1-3 substituents chosen from alkyl, carboxy, —COOR₂ (wherein R₂ is the same as defined earlier), hydroxy, alkoxy, halogen, —CF₃, cyano, —NRₓR_{y}, —C(═O)NRₓR_{y}, —OC(═O)NRₓR_{y} (wherein Rₓ, and R_{y} are the same as defined earlier) and —S(O)ₙR₃ (where R₃ and n are the same as defined earlier).

The term “alkynyl,” unless otherwise specified, refers to a monoradical of an unsaturated hydrocarbon, having from 2 to 20 carbon atoms. In the event that alkynyl is attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, aminocarbonylamino, nitro, heterocyclyl, heteroaryl, heterocyclylalkyl, heteroarylalkyl, —NHC(═O)R_{f}, —NR_{f}R_{q}, —NHC(═O)NR_{f}R_{q}, —C(═O)NR_{f}R_{q}, —O—C(═O)NR_{f}R_{q} (wherein R_{f} and R_{q} are the same as defined earlier), or —SO₂R₆ (wherein R₆ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NR_{f}R_{q}, —C(═O)NR_{f}R_{q}, —NHC(═O)NR_{f}R_{q}, —C(═O)NR_{f}R_{q} (wherein R_{f} and R_{q} are the same as defined earlier), cyano, or —SO₂R₆ (where R₆ is same as defined earlier).

The term “alkynylene” unless otherwise specified, refers to a diradical of a triply-unsaturated hydrocarbon, preferably having from 2 to 6 carbon atoms. In the event that alkynylene is attached to the heteroatom, the triple bond cannot be alpha to the heteroatom.

The alkenylene group can be connected by two bonds to the rest of the structure of compound of Formula I. Alkynylene may further be substituted with one or more substituents such as alkyl, alkenyl, alkoxy, cycloalkyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, nitro, heterocyclyl, heteroaryl, heterocyclyl alkyl, heteroarylalkyl, —NHC(═O)Rₓ, —NRₓR_{y}, —NHC(═O)NRₓR_{y}, —C(═O)NRₓR_{y}, —OC(═O)NRₓR_{y} (wherein Rₓ, and R_{y} are the same as defined earlier), —S(O)ₙR₃ (where R₃ and n are the same as defined earlier). Unless otherwise constrained by the definition, all substituents may optionally be further substituted by 1-3 substituents chosen from alkyl, carboxy, —COOR₂ (wherein R₂ is the same as defined earlier), hydroxy, alkoxy, halogen, CF₃, —NRₓR_{y}, —C(═O)NRR_{y}, —NHC(═O)NRₓR_{y}, —C(═O)NRₓR_{y} (wherein Rₓ and R_{y} are the same as defined earlier), cyano, and —S(O)ₙR₃ (where R₃ and n are the same as defined earlier).

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl, and bicyclo [2.2.1]heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{f}R_{q}, —NHC(═O)NR_{f}R_{q}, —NHC(═O) R_{f}, —C(═O)NR_{f}R_{q}, O—C (═O)NR_{f}R_{q} (wherein R_{f} and R_{q} are the same as defined earlier), nitro, heterocyclyl, heteroaryl, heterocyclylalkyl, heteroarylalkyl, or SO₂—R₆ (wherein R₆ is same as defined earlier). Unless otherwise constrained by the definition, cycloalkyl substituents optionally may be substituted further by 1-3 substituents selected from alkyl, carboxy, hydroxy, alkoxy, halogen, CF₃, —NR_{f}R_{q}, C(═O)NR_{f}R_{q}, —NHC(═O)NR_{f}R_{q}, —OC(═O)NR_{f}R_{q} (wherein R_{f} and R_{q} are the same as defined earlier), cyano or —SO₂R₆ (where R₆ is same as defined earlier). “Cycloalkylalkyl” refers to alkyl-cycloalkyl group linked through alkyl portion, wherein the alkyl and cycloalkyl are the same as defined earlier.

As used herein the term “halogen or halo” refers to fluorine, chlorine, bromine or iodine.

As used herein the term “hydroxyl-protecting group” includes, but are not limited to, acyl, aroyl, alkyl, aryl, butyldiphenylsilyl, methoxymethyl and methylthiomethyl, and the like.

As used herein the term “thio” refers to the group —SH.

As used herein the term “alkoxy” stands for a group O—R wherein R refers to alkyl or cycloalkyl. Examples of alkoxy include, but are not limited to, methoxy, ethoxy, cyclopentoxy and the like.

As used herein the term “thioalkyl” refers to —SR wherein R refers to alkyl or cycloalkyl.

As used herein the term “haloalkyl” refers to alkyl of which one or more hydrogen (s) is/are replaced by halogen.

The term “aryl,” unless otherwise specified, refers to carbocyclic aromatic groups, for example, phenyl, biphenyl or napthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₑ (wherein R_{c} is hydrogen, alkyl, alkenyl, cycloalkyl, aralkyl, heterocyclylalkyl, heteroarylalkyl), NHC(═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), —SO₂R₆ (wherein R₆ is same as defined earlier), carboxy, heterocyclyl, heteroaryl, heterocyclylalkyl, heteroarylalkyl or amino carbonyl amino. The aryl group optionally may be fused with a cycloalkyl group, wherein the cycloalkyl group may optionally contain heteroatoms selected from O, N or S.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl and the like.

The term “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, aryl, alkoxy, alkaryl, cyano, nitro, oxo, carboxy, heterocyclyl, heteroaryl, —O—C(═O)R_{f}, —O—C(═O)OR_{f}, —C(═O)NR_{f}R_{q}, SO₂R₆, —O—C(═O)NR_{f}R_{q}, —NHC(═O)NR_{f}R_{q}, —NR_{f}R_{q} (wherein R₆, R_{f} and R_{q} are as defined earlier) or guanidine. Heterocyclyl can optionally include rings having one or more double bonds. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, dihydropyridinyl, dihydroisoxazolyl, dihydrobenzofuryl, azabicyclohexyl, dihydroindolyl, pyridinyl, isoindole 1,3-dione, piperidinyl or piperazinyl azetidinyl, benzimidazolyl, 1,4-benzodioxanyl, 1,3-benzodioxolyl, benzoxazolyl, benzothiazolyl, benzothienyl, benzotriazolyl, dihydroimidazolyl, dihydropyranyl, dihydrofuranyl, dioxanyl, dioxolanyl, furyl, homopiperidinyl, imidazolyl, imidazolinyl, imidazolidinyl, indolinyl, indolyl, isoquinolinyl, isothiazolidinyl, isothiazolyl, isoxazolidinyl, isoxazolyl, morpholinyl, napthyridinyl, oxazolidinyl, oxazolyl, piperazinyl, piperidinyl, purinyl, pyrazinyl, pyrazolinyl, pyridinyl, pyrimidinyl, pyrrolidinyl, pyrrolinyl, pyrrolyl, pyrrolopyridinyl, imidazolpyridinyl, quinolinyl, tetrahydrofuranyl, tetrahydropyranyl, thiazolidinyl, thiazolyl, thienyl, and the like.

As used herein the term “(heterocycle) alkyl” stands for heterocycle, which is bonded to an alkylene chain. Examples of heterocycle alkyl include, but are not limited to, isothiazolidinyl ethyl, isothiazolyl propyl, pyrazinyl methyl, pyrazolinyl propyl and pyridyl butyl, and the like.

The aryl and heterocycle may optionally be substituted with one or more substituent(s) independently selected from the group consisting of hydroxy, halogen, nitro, mercapto, cyano, alkyl, haloalkyl, alkoxy, thioalkyl, optionally substituted aryl, optionally substituted heterocyclyl, —NHCONHR⁹, wherein R⁹ can be alkyl, alkenyl, alkynyl, cycloalkyl, aryl, aralkyl, (heterocycle)alkyl or heterocyclyl, —NR¹¹R¹², —CONR¹¹R¹², —COOR¹¹, —CONHR¹¹, —OCOR¹¹, —COR¹¹, —NHSO₂R¹¹, and —SO₂NHR¹¹, wherein R¹¹ and R¹² are independently selected from the group comprising of hydrogen and alkyl.

As used herein the term “polymorphs” includes all crystalline forms and amorphous forms for compounds described herein. In addition, some of the compounds described herein may form solvates with water (i.e. hydrate, hemihydrate or sesquihydrate) or common organic solvents. Such solvates are also encompassed within the scope of this invention.

The phrase “pharmaceutically acceptable salts” denotes salts of the free base, which possess the desired pharmacological activity of the free base and which are neither biologically nor otherwise undesirable. Suitable pharmaceutically acceptable salts may be prepared from an inorganic or organic acid. Examples of such inorganic acids include, but are not limited to, hydrochloric, hydrobromic, hydroiodic, nitrous (nitrile salt), nitric (nitrate salt), carbonic, sulfuric, phosphoric acid and like. Appropriate organic acids include, but are not limited to, aliphatic, cycloaliphoric, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumeric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, beta-hydroxybutyric, cyclohexylaminosulfonic, galactaric and galacturonic acid and the like.

The term “pharmaceutically acceptable solvates” refers to solvates with water (i-e hydrates) or pharmaceutically acceptable solvents, for example solvates with ethanol and the like. Such solvates are also encompassed within the scope of the disclosure. Furthermore, some of the crystalline forms for compounds described herein may exist as polymorphs and as such are intended to be included in the scope of the disclosure.

The present invention also includes within its scope prodrugs of these agents. In general, such prodrugs will be functional derivatives of these compounds, which are readily convertible in vivo into the required compound. Conventional procedures for the selection and preparation of suitable prodrug derivatives are described, for example, in “Design of Prodrugs”, ed. H Bundgaard, Elsevier, 1985. The disclosed compounds may get metabolized in vivo and these metabolites are also encompassed within the scope of this invention.

The compounds of present invention include stereoisomers. The term “stereoisomer” refers to compounds, which have identical chemical composition, but differ with regard to arrangement of the atoms and the groups in space. These include enantiomers, diastereomers, geometrical isomers, atropisomer and comformational isomers. Geometric isomers may occur when a compound contains a double bond or some other feature that gives the molecule a certain amount of structural rigidity. An eanantiomer is a stereoisomer of a reference molecule that is the nonsuperimposable mirror image of the reference molecule. A diastereomer is a stereoisomer of a reference molecule that has a shape that is not the mirror image of the reference molecule. An atropisomer is a conformational of a reference compound that converts to the reference compound only slowly on the NMR or laboratory time scale. Conformational isomers (or conformers or rotational isomers or rotamers) are stereoisomers produced by rotation about a bonds, and are often rapidly interconverting at room temperature. Racemic mixtures are also encompassed within the scope of this invention.

There may be tautomers for some of the disclosed compounds, and the present invention covers all the possible isomers including tautomers and mixtures thereof.

Compounds disclosed herein may be prepared by techniques well known to one of ordinary skill in the art. In addition, compounds of disclosed herein may also be prepared by the following reaction sequences as depicted in Schemes I, II and III below.

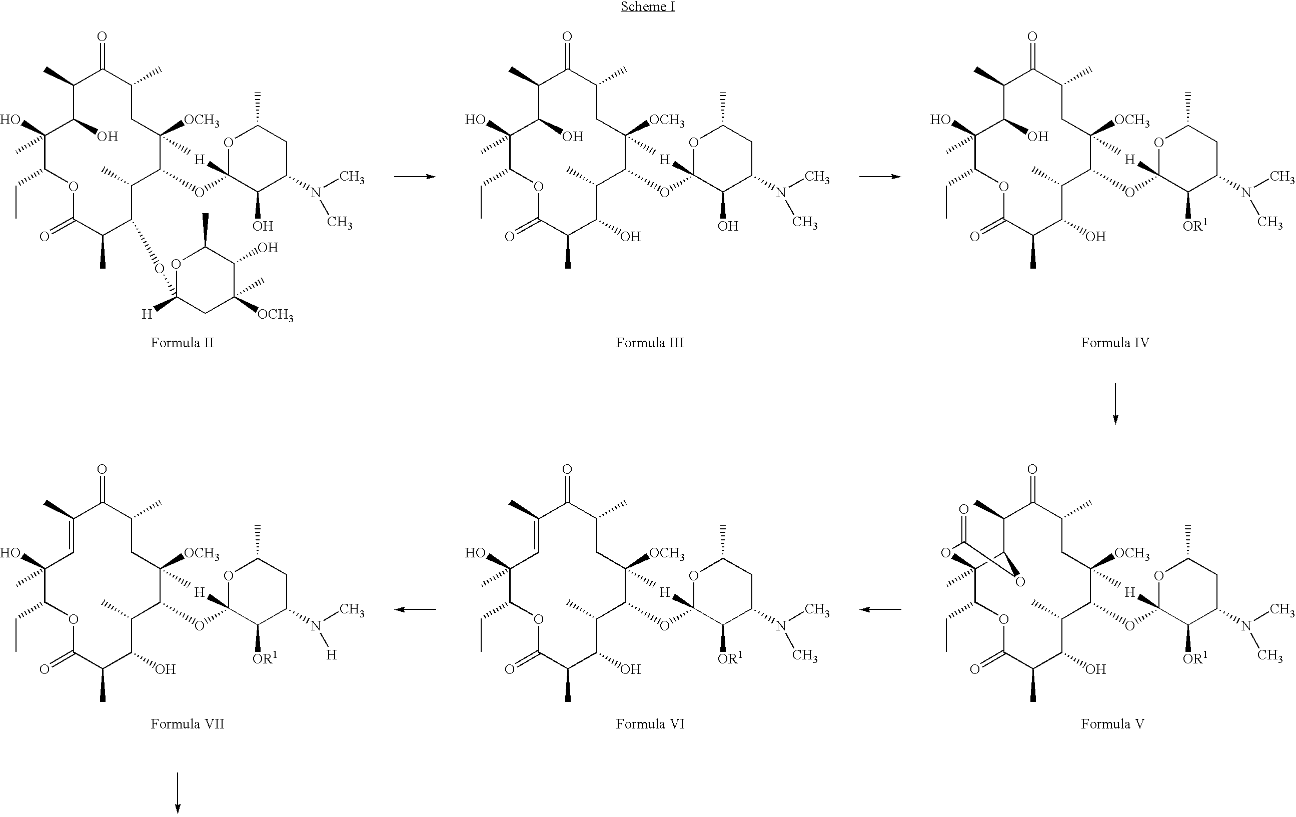

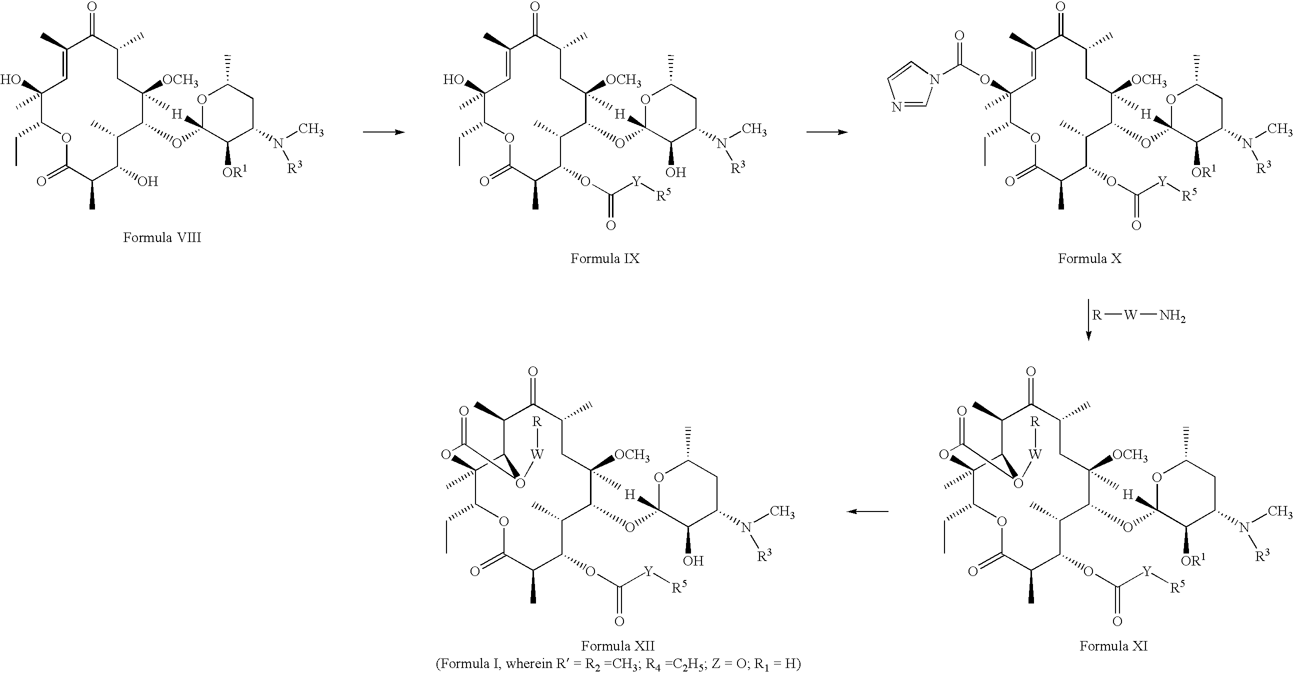

Compounds of Formula XII can be prepared according to Scheme I. Thus, clarithromycin of Formula II can be hydrolyzed to form compounds of Formula III. Compounds of Formula III can be protected with one or more reagents of Formula R¹₂O or R¹X (wherein X is halogen) to form compounds of Formula IV. Compounds of Formula IV can be reacted with one or more reagents to form compounds of Formula V. Compounds of Formula V can be reacted with one or more organic bases to from compounds of Formula VI. Compounds of Formula VI can be desmethylated to from compounds of Formula VII. Compounds of Formula VII can be alkylated with one or more reagents of Formula R³CHO, R³₂CO or R³X to form compounds of Formula VIII (wherein R³ is the same as defined earlier). Compounds of Formula VIII can be acylated with one or more reagents of Formula R⁵YCOOH, (R⁵YCO)₂O, R⁵YCOX or R⁵YCOOR¹⁰ (wherein R¹⁰ is a leaving group, for example, pivaloyl, p-toleuensulfonyl, isobutoxycarbonyl, ethoxycarbonyl or isopropoxycarbonyl) to form compounds of Formula IX (wherein Y and R⁵ are the same as defined earlier). Compounds of Formula IX can be reacted with N,N′-carbonyl diimidazole to form compounds of Formula X. Compounds of Formula X can be reacted with compounds of Formula R—W—NH₂ to form compounds Formula XI (wherein R and W are the same as defined earlier). Compounds of Formula XI can be deprotected to form compounds of Formula XII.

Clarithromycin of Formula II can be hydrolyzed in the presence of one or more inorganic or organic acid, for example, hydrochloric acid, sulphuric acid, dichloroacetic acid or mixture thereof, in one or more solvents, for example, dichloromethane, dichloroethane, carbon tetrachloride, chloroform, ethyl acetate or mixture thereof.

The compounds of Formula III can be hydroxyl protected by reacting with one or more reagents of Formula R¹₂O or R¹X in one or more solvents, for example, dichloromethane, dichloroethane, chloroform, carbon tetrachloride, ethyl acetate or mixture thereof. The protection reaction can also be carried out in the presence of one or more organic bases, for example, triethylamine, pyridine, tributylamine, 4-N-dimethylaminopyridine, diisopropyl ethyl amine, or mixture thereof.

Compounds of Formula IV can be reacted with one or more reagents, for example, triphosgene or ethylene carbonate, in one or more solvents, for example, chloroform, dichloromethane, carbon tetrachloride, dichloroethane or mixture thereof. The reaction can also be carried out in the presence of one or more organic bases, for example, triethylamine, pyridine, tributylamine, 4-N-dimethylaminopyridine, diisopropyl ethyl amine or mixtures thereof.

Compounds of Formula V can be reacted with one or more organic bases, for example, tetramethyl guanidine, pyridine, trimethylamine, diisopropyl ethyl amine or mixtures thereof, in one or more solvents, for example, dimethylformamide, tetrahydrofuran, dimethylsulfoxide or mixtures thereof.

Compounds of Formula VI can be desmethylated in the presence of one or more demethylating agents, for example, N-iodosuccinamide, iodine in acetic acid, diisopropyl azodicarboxylate or mixtures thereof. The reactions can also be carried out in one or more solvent, for example, acetonitrile, tetrahydrofuran, dichloromethane, dichloroethane, chloroform, carbon tetrachloride, ethyl acetate or mixtures thereof.

The desmethylation reaction can be quenched by the presence of one or more quenching agents, for example, sodium bisulphite, potassium carbonate, sodium acetate, sodium carbonate or mixtures thereof.

Compounds of Formula VII can be alkylated with one or more reagents of Formula R³CHO, R³₂CO or R³X in one or more solvents, for example, dimethylformamide, acetonitrile tetrahydrofuran or mixtures thereof. The alkylation reaction can also be carried out in presence of one or more organic or inorganic bases, for example, sodium hydrogen carbonate, potassium carbonate, sodium acetate, sodium thiosulfate, sodium hydride, pyridine, triethylamine, diisopropyl ethyl amine or mixture thereof. The alkylation reaction can also be carried out in presence of one or more reducing agents, for example, sodium cyanoborohydride, sodium borohydride or sodium triacetoxyborohydride or mixtures thereof, in the presence of one or more organic acids, for example, acetic acid, dichloroacetic acid or mixtures thereof.

Compounds of Formula VIII can be acylated with one or more reagents of Formula R⁵YCOOH, (R⁵YCO)₂O, R⁵YCOX or R⁵YCOOR¹⁰ (wherein R¹⁰ is as defined earlier) in one or more solvents, for example, dichloromethane, dichloroethane, acetone, chloroform, carbon tetrachloride, ethyl acetate, tetrahydrofuran or mixtures thereof. The acylation reaction can also be carried out in the presence of one or more inorganic or organic bases, for example, sodium bicarbonate, potassium carbonate, triethylamine, pyridine, tributylamine, 4-N-dimethylaminopyridine, diisopropyl ethyl amine or mixtures thereof. This reaction can also be carried out in the presence of one or more activating agents, for example, N,N′-dicyclohexyl carbodiimide, 1-ethyl-3-(3-dimethylaminopropyl) carbodiimide hydrochloride or mixtures thereof.

Compounds of Formula IX can be reacted with N,N′-carbonyldiimidazole in one or more solvents, for example, dimethylformamide, tetrahydrofuran or mixtures thereof. The reaction can also be carried out in the presence of one or more inorganic bases, for example, sodium hydrogen carbonate, potassium carbonate, diisopropyl ethyl amine, sodium hydride or mixtures thereof.

Compounds of Formula X can be reacted with compounds of Formula R—W—NH₂ in one or more solvents, for example, acetonitrile, water, dimethylformamide or mixtures thereof.

Compounds of Formula XI can be deprotected in one or more alcoholic solvents, for example, methanol, ethanol, propanol, isopropanol or mixtures thereof.

Compounds of Formula XII can further be converted into their pharmaceutically acceptable salts by following conventional methods known to one ordinary skill in the art.

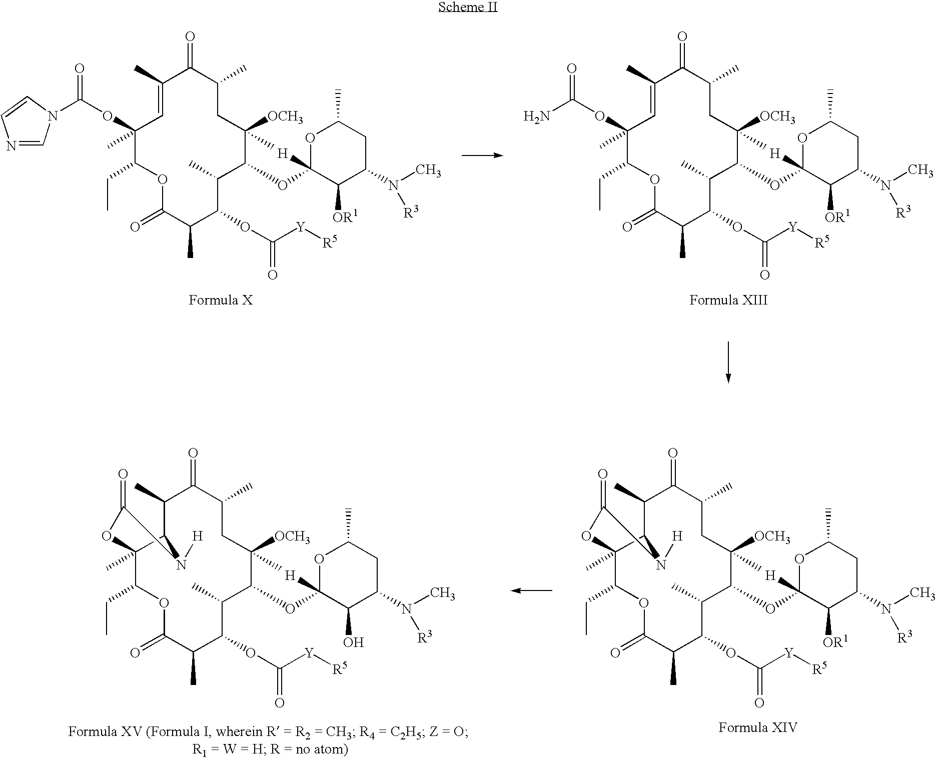

Compounds of Formula XV can be prepared according to Scheme II. Thus, compounds of Formula X can be reacted with ammonia to form compounds of Formula XIII (wherein R³, R¹, Y and R⁵ are the same as defined earlier). Compounds of Formula XIII can be cyclized to form compounds of Formula XIV. Compounds of Formula XIV can be deprotected to form compounds of Formula XV.

Compound of Formula X can be reacted with ammonia in one or more solvents, for example, acetonitrile, water, dimethylformamide or mixture thereof.

Compounds of Formula XIII can be cyclized in one or more solvents, for example, dichloromethane, dichloroethane, acetone, dimethylormamide, ethyl acetate, tetrahydrofuran or mixtures thereof. The reaction can also be carried out in presence of one or more organic bases, for example, n-butyl lithium lithium-3-aminopropanamide, alkali-alkoxides (e.g., potassium tert-butoxide) or mixtures thereof.

Compound of Formula XIV can be deprotected in one or more alcoholic solvents, for example, methanol, ethanol, propanol or isopropanol.

Compounds of Formula XV can further be converted into their pharmaceutically acceptable salts by following conventional methods known to one ordinary skill in the art.

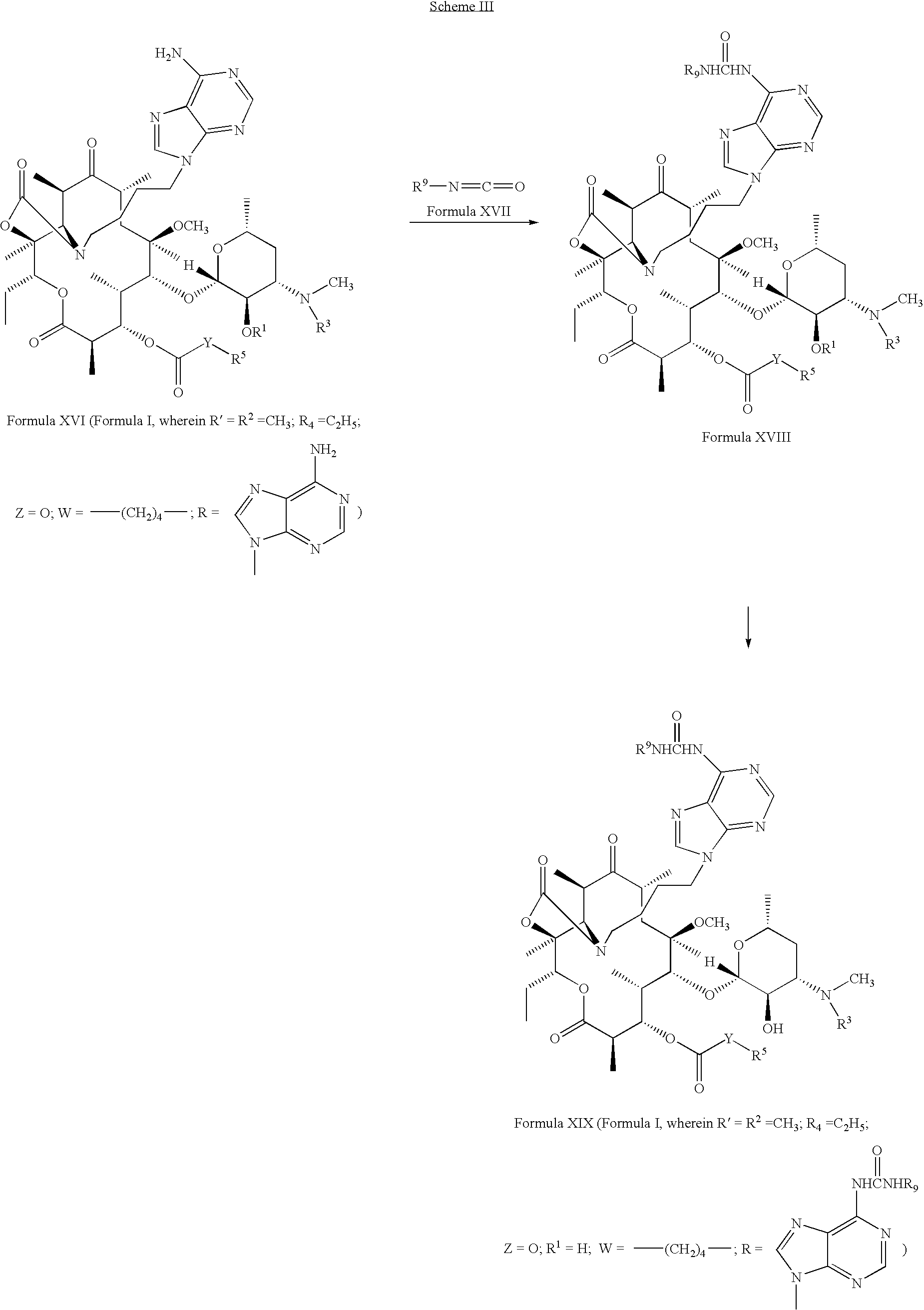

Compounds of Formula XIX can be prepared according to Scheme III. Thus, compounds of Formula XVI can be reacted with compounds of Formula XVII to form compounds of Formula XVIII (wherein R¹, R⁹, R³, Y and R⁵ are the same as defined earlier). Compounds of Formula XVIII can be deprotected to form compounds of Formula XIX.

Compounds of Formula XVI can be reacted in one or more solvents, for example, dichloromethane, dichloroethane, acetone, ethyl acetate, chloroform, carbon tetrachloride, tetrahydrofuran or mixtures thereof.

Compounds of Formula XVIII can be deprotected in one or more alcoholic solvents, for example, methanol, ethanol, propanol or isopropanol.

Compounds of Formula XIX can further be converted to their pharmaceutically acceptable salts by following conventional methods known to one ordinary skill in the art.

In the above scheme, where the specific bases, activating agents, solvents, etc., are mentioned, it is to be understood that bases, activating agents, solvents, etc., known to one ordinary skill in the art may be used. Similarly, the reaction temperature and duration may be adjusted according to the desired needs without undue experimentation and well within the abilities of one of ordinary skill in the art.

Representative compounds useful for such purposes are listed below:
11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-(benzoimidazol-1-yl)-pentyl)-imino)]erythromycin A (Compound No. 1),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(benzoimidazol-1-yl)-pentyl)-imino)]erythromycin A (Compound No. 2),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(1H-imidazo[4,5-b]pyridin-1-yl)-pentyl)-imino)]erythromycin A (Compound No. 3),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(3H-imidazo[4,5-b]pyridin-3-yl)-pentyl)-imino)]erythromycin A (Compound No. 4),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-pyridin-3-yl-phenoxy)-imino)]erythromycin A (Compound No. 5),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((1-(9-(4-amino-butyl)-9H-purin-6-yl)-3-butyl)-urea)-imino)]erythromycin A (Compound No. 6),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((1-(9-(4-amino-butyl)-9H-purin-6-yl)-3-(2,6-difluoro-phenyl)-urea)-imino)]erythromycin A (Compound No. 7),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((1-allyl-3-(9-(4-amino-butyl)-9H-purin-6-yl)-urea)-imino)]erythromycin A (Compound No. 8),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((1-(9-(4-amino-butyl)-9H-purin-6-yl)-3-(4-fluoro-phenyl)-urea)-imino)]erythromycin A (Compound No. 9),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(3-pyridin-3-yl-phenoxy)-propyl)-imino)]erythromycin A (Compound No. 10),
11,12-dideoxy-3-O-decladinosyl-3-O-(2-pyridyl acetyl)-5-O-(3′-N-desmethyl-3′-N-allyl)-6-O-methyl-12,11-[oxycarbonyl-imino)]erythromycin A (Compound No. 11),
11,12-dideoxy-3-O-decladinosyl-3-O-(3-pyridyl acetyl)-5-O-(3′-N-desmethyl-3′-N-allyl)-6-O-methyl-12,11-[oxycarbonyl-imino)]erythromycin A (Compound No. 12),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(3-thiophen-3-yl-phenoxy)-propyl)-imino)]erythromycin A (Compound No. 13),

their pharmaceutically acceptable salts, pharmaceutically acceptable solvates, stereoisomers, tautomers, racemates, prodrugs, metabolites and polymorphs.

The compounds disclosed herein are pharmacologically active against Gram-positive, Gram-negative and anaerobic bacteria and accordingly, are useful as antibacterial agents for treating bacterial infections in a patient in need thereof, for example, in a human or an animal. provided herein are pharmaceutical compositions, which may be administered to an animal for treatment orally, topically, rectally, internasally, or by a parenteral route. The pharmaceutical compositions of disclosed compounds comprise a pharmaceutically effective amounts of compounds described herein formulated together with one or more pharmaceutically acceptable carriers. “The term pharmaceutically acceptable carriers” is intended to include non-toxic, inert solid, semi-solid or liquid filler, diluent, encapsulating material or formulation auxiliary of any type.

Solid form preparations for oral administrations include capsules, tablet, pills, powder, granules, cachets and suppository. For solid form preparation, active compounds can be mixed with one or more inert, pharmaceutically acceptable excipients or carriers, for example, sodium citrate, dicalcium phosphate and/or a filler or extenders (e.g., starches, lactose, sucrose, glucose, mannitol and silicic acid or mixture thereof); binders (e.g., carboxymethylcellulose, alginates, gelatins, polyvinylpyrrolidinone, sucrose, acacia or mixture thereof); disintegrating agents (e.g., agar-agar, calcium carbonate, potato starch, alginic acid, certain silicates, sodium carbonate or mixture thereof); absorption acceletors (e.g., quaternary ammonium compounds); wetting agents (e.g., cetyl alcohol, glycerol mono stearate or mixture thereof); adsorbants (e.g., Kaolin); lubricants (e.g., talc, calcium stearate, magnesium stearate, solid polyethyleneglycol, sodium luaryl sulphate or mixture thereof).

Capsules, tablets, pills may also comprise bufferring agents. Tablets, capsules, pills or granules can be prepared with one or more coating and shells, for example, enteric coating and other coatings well known to one ordinary skill in the art.

Liquid form preparations for oral administration include pharmaceutically acceptable emulsions, solutions, suspensions, syrups and elixirs. In such liquid form preparations, active compounds can be mixed with water or one or more other solvents, solubilizing agents or emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, for example, cottonseed, groundnut, corn, germ, olive, castor and sesame oil), glycerol, fatty acid esters of sorbitan or mixture thereof. Oral compositions can also include one or more adjuvants, for example, wetting agents, emulsifying agents, suspending agents, sweetening agents, flavoring agents, perfuming agents or mixture thereof.

Injectable preparations, for example, sterile injections, aqueous suspensions may be formulated according to methods known to one of ordinary skill in the art, and in particular using suitable dispersing or wetting and suspending agents. Acceptable vehicles and solvents that may be employed include water, Ringer's solution, isotonic sodium chloride or mixture thereof.

Dosage forms for tropical or transdermal administration of a compound of the present invention include ointments, pastes, creams, lotions, gels, powders, solutions, sprays, inhalants or patches. Active compound can be admixed under sterile condition with one or more pharmaceutically acceptable carriers and optionally any preservatives or buffers as may be required. Ophthalmic formulations, eardrops, eye ointments, powder and solution are also encompassed within the scope of this invention.

Pharmaceutical preparations may be in unit dosage form. In unit dosage form, the preparations can be subdivided into unit doses containing appropriate quantities of the active components. Unit dosage forms can be packaged preparations containing discrete capsules, powders, in vials or ampoules, ointments, capsules, sachets, tablets, gels, creams or any combination and number of such packaged forms.

Quantity of active compound in unit dose of preparation may be varied or adjusted from less than 1 mg to several grams according to the particular application and potency of the active ingredients.

In therapeutic use as agents for treating bacterial infections the compounds utilizing in the pharmaceutical method of this invention can be administered at the initial dosage of about 3 mg to about 40 mg per kilogram daily. The dosages, however, may be varied depending upon the requirements of the patients and the compound being employed.

Determination of the proper dosage for a particular situation can be within the smaller dosages, which may be less than the optimum dose. Small increments until the optimum effect under the daily dosage may be divided and administered in portion during the day if desired.

While the present invention has been described in terms of its specific embodiments, certain modifications and equivalents will be apparent to those skilled in the art and included within the scope of the present invention. The examples are provided to illustrate particular aspects of the disclosure and do not constrain the scope of the present invention as defined by the claims.

### EXAMPLES

### Example 1

### Preparation of R—W—NH₂

Step (a) Preparation of 9-(4-aminobutyl)-9H-purin-6-amine [W is —(CH₂)₄]

9-(4-aminobutyl)-9H-purin-6-amine was prepared according to a procedure disclosed in U.S. Pat. No. 5,635,485. In particular, 10.3 g of potassium carbonate were added to a solution of 5.95 g of 9H-purin-6-amine and 15.5 g of N-4-bromobutyl-phthalimide in 30 mL of dimethylformamide and the mixture was stirred for 20 hours at ambient temperature. The insoluble part was filtered off and rinsed with methylene chloride. The organic phase was washed with water, then dried over magnesium sulphate and evaporated. The oily residue obtained was washed with petroleum ether, then with isopropyl ether to obtain 16.3 g of a yellow solid, which was purified by chromatography on silica, eluting with a methylene chloride: acetone mixture to obtain 4.9 g of product (A) melting at 143° C.

A mixture of 32.86 g of product (A) obtained above, 697 mL of ethanol and 20 mL of hydrazine was refluxed for 19 hours. The mixture was allowed to return to ambient temperature, filtered, rinsed and evaporated to dryness. The residue was taken up in methylene chloride, filtered, rinsed and evaporated to dryness to obtain 18.87 g of the desired product.

Step (b) Preparation of 4-(3H)-imidazo[4,5-b]pyridin-3-yl) pentan-1-amine [W is —(CH₂)₃—CH(CH₃)—]

Step 1: Preparation of 5-(tert-butyl-dimethyl-silanyloxy)pentan-2-ol

1,4 pentanediol (5 g, 48.0 mmoles, 1.0 eq) was taken in dimethylformamide (10 ml) and cooled to 0° C. To it imidazole (4.9 g, 72 mmole, 1.5 eq) was added and reaction mixture was stirred for about 10 minutes followed by the addition of tert-butyl-dimethyl-silanyloxy chloride (8.0 g, 52.8 mmoles, 1.1 eq) in dimethylformamide (5 ml). The reaction mixture was the allowed to stir at room temperature for about 20 hour, poured into ice-cold water and extracted with ethyl acetate. Evaporation of solvent gave the product. Yield: 10.6 g

Step 2: Preparation of methanesulphonic acid-4-(tert-butyl-dimethyl-silanyloxy)-1-methyl-butyl ester

5-(tert-butyl-dimethyl-silanyloxy)pentan-2-ol (10.5 g, 47.9 mmoles, 1.0 eq.) was taken in dichloromethane (20 ml) and cooled to 0° C. To this triethyl amine (10 ml, 71.9 mmole, 1.5 eq.) was added and the reaction mixture was stirred for about 10 min followed by the addition of methane sulphonyl chloride (4.5 ml, 57.5 mmoles, 1.2 eq). The reaction mixture was stirred for about 1 hour at 0° C., quenched by addition of aqueous sodium bicarbonate solution and extracted with dichloromethane. Evaporation of dichloromethane layer gave the product. Yield: 12.8 g

Step 3: Preparation of 3-(4-{[tert-butyl(dimethyl)silyl]oxy}-1-methylbutyl)-3H-imidazo[4,5-b]pyridine (isomer [a]) and 1-(4-{[tert-butyl(dimethyl)silyl]oxy}-1-methylbutyl)-1H-imidazo[4,5-b]pyridine (isomer [b])

3H-imidazo[4,5-b]pyridine (3.5 g, 29.4 mmol, 1.0 eq) was taken in dimethylformamide (10 ml) and cooled to 0° C. To this sodium hydride (1.8 g, 44.1 mmol, 1.5 eq) was added in portions and after the additions were over, the contents were stirred for about 15 min. Then methanesulphonic acid-4-(tert-butyl-dimethyl-silanyloxy)-1-methyl-butyl ester (11.4 g, 38.2 mmol, 1.3 eq) in dimethylformamide (5 ml) was added at 0° C. and the reaction mixture was allowed to come to room temperature and stirred for about 24 hour, poured into ice-cold water and extracted with ethyl acetate. The organic layer was washed with water and dried with brine and sodium sulphate and concentrated under reduced pressure to get the crude product. It was then purified by column chromatography using 100-200 mesh silica gel and hexane: acetone: triethyl amine to get the two isomers. Yield: isomer [a]: 2.5 g; isomer [b]: 1.5 g

Step 4: Preparation of 4-(3H-imidazo[4,5-b]pyridin-3-yl) pentan-1-ol

To the 3-[4-(tert-butyl-dimethyl-silanyloxy)-1-methyl-butyl]-3H-imidazo[4,5-b]pyridine (2.4 g, 0.0075 moles, 1 eq.) was added tetra-n-butylamonium fluoride (3.5 ml, 0.0135 mol, 1.8 eq.) and the reaction mixture was heated at 55° C. for about 45 minutes. The reaction mixture was poured in water and extracted with ethyl acetate, washed with water, brine and dried over anhydrous sodium sulphate. The organic layer was concentrated and vacuum dried. Yield: 1.4 g

Step 5: Preparation of methane sulphonic acid 4-(3H)imidazo[4,5-b]pyridin-3-yl-pentyl ester

To 4-(3H-imidazo[4,5-b]pyridin-3-yl) pentan-1-ol (3 g, 0.0146 moles, 1 eq.) was added dichloromethane (10 ml) and it was cooled to 0° C. Tri ethyl amine (3 ml, 0.0219 moles, 1.5 eq) was added to it followed by methane sulphonyl chloride (1.35 ml, 0.01752 moles, 1.2 eq.). The reaction mixture was stirred for about 45 minutes, quenched with aqueous sodium bicarbonate solution and extracted with dichloromethane. The organic layer was washed with water, brine and dried over anhydrous sodium sulphate. The organic layer was concentrated and vacuum dried. Yield: 3.2 g

Step 6: Preparation of 2-{4-(3H-imidazo[4,5-b]pyridin-3-yl-pentyl)-isoindole-1,3-dione

To phthalimide (1.1 g, 0.0074 mole, 1 eq.) in dry dimethylformamide (5 ml) was added sodium hydride (0.448 g, 0.01122 moles, 1.5 eq.) in portions. After about 20 minutes, methane sulphonic acid 4-3H)— imidazo[4,5-b]pyridin-3-yl-pentyl ester (3.2 g, 0.01122 moles, 1.5 eq.) dissolved in dry dimethylformamide (5 ml) was added. It was allowed to stir for about 14 hours. The reaction mixture was poured in ice-cold water and extracted with ethyl acetate. The organic layer was washed with water, brine and dried over anhydrous sodium sulphate. Yield=2 g

Step 7: Preparation of 4-(3H-imidazo[4,5-b]pyridin-3-yl)pentan-1-amine

2-[4-(3H-imidazo[4,5-b]pyridin-3-yl-pentyl)-isoindole-1,3-dione (2 g, 5.98 mmoles, 1 eq) was taken in ethanol (20 ml) and hydrazine monohydrate (6 ml, 11.9 mmol, 2 eq) was added to it. The reaction mixture was heated at 70° C. for about 4 hours. It was cooled to room temperature and solid obtained was filtered through celite bed. The filtrate was concentrated to get the crude product, which was purified over 100-200 mesh silica gel column using dichloromethane and methanol as eluant. Yield: 0.22 g, 36%

The following illustrative compound was prepared analogously by following the above procedure:

4-(1H-benzimidazol-1-yl) pentan-1-amine.
Step (c) Preparation of 3-(4-pyridin-3-yl phenoxy) propan-1-amine [W═—(CH₂)₃—O—]
Step 1: Preparation of pyridin-3-boronic acid

3-bromo pyridine (5 g) was dissolved in dry tetrahydrofuran (20 ml), cooled to −78° C. and to it tri-isopropyl borate (14.68 ml) was added, followed by addition of butyl lithium (15% in hexane, 21 ml). The reaction mixture was stirred at −78° C. for about 4 hours, pH was adjusted to about 7, it was extracted with ethyl acetate and concentrated to get white solid.

Yield: 1.2 g

Step 2: Preparation of 2-[3-(3-bromo-phenoxy)-propyl]-isoindole-1,3-dione

To 3-bromo phenol (5.5 g, 0.03178 moles, 1 eq), dry dimethylformamide (30 ml) was added. It was cooled to 0° C. Then sodium hydride (1.9 g, 0.0476 moles, 1.5 eq) was added in portions. After 30 minutes, N-3-bromopropyl phthalimide (10.2 g, 0.0381 moles, 1.2 eq) was added. The reaction mixture was stirred for about 3 hours. It was quenched by pouring reaction mixture into ice cold water. The precipitate was filtered and vacuum dried. Yield: 9 g.

Step 3: Preparation of 2-{3-[3-(pyridin-3-yl)-phenoxy]-propyl}-isoindole-1,3-dione

Pyridin-3-boronic acid (0.75 g, 6.11 mmoles, 1.1 eq.), 2-[3-(3-bromo-phenoxy)-propyl]-isoindole-1,3-dione (2 g, 5.5 mmoles, 1 eq.) and potassium carbonate (3 g, 22.2 mmoles, 4 eq.) were taken in a round bottom flask and degassed for about 1 hour. Dry dimethylformamide (15 ml) was added. The reaction mixture was than flushed with argon for about 15 minutes. Tetrakis (triphenylphosphine) palladium (0) (0.32 g, 0.27 moles, 0.05 eq.) was added to it. The reaction mixture was heated at 80° C. for about 12 hours. It was quenched with water and extracted with ethyl acetate. The organic layer was washed with water, brine and dried over anhydrous sodium sulphate. Yield=0.2 g

Step 4: Preparation of 3-(4-pyridin-3-yl phenoxy)propan-1-amine

2-{3-[3-(pyridin-3-yl)-phenoxy]-propyl}-isoindole-1,3-dione (0.2 g) was taken in ethanol (10 ml) and hydrazine monohydrate (0.2 ml) was added to it. The reaction mixture was heated at 60° C. for about 4 hours. It was cooled to room temperature and solid obtained was filtered through celite bed. The filtrate was concentrated to get the crude product, which was purified using dichloromethane and methanol as eluant. Yield: 0.13 g

The following illustrative compounds were prepared analogously following the above procedure:

3-[3-(3-thienyl)phenoxy]propan-1-amine
3-(3-pyridin-3-yl-phenoxy) propan-1-amine.

### Example 2

### Synthetic Procedure of Scheme I

Step (a) Preparation of compound of Formula III

Clarithromycin (25 g, 33.4 mmol) was added to a solution of hydrochloric acid (1N, 250 ml) at ambient temperature. The reaction mixture was neutralized with solid sodium bicarbonate and the aqueous layer was extracted with ethyl acetate. The organic layer was washed successively with water and then brine, and dried over anhydrous sodium sulphate and the solvent was removed under reduced pressure to yield the title compound. The crude product was crystallized using ethyl acetate-hexane mixture.

### Step (b) Preparation of Compound of Formula IV

Benzoic anhydride (2.5 equiv.) followed by triethylamine (6 equiv) was added to a solution of compound of Formula III (1 equiv) in dry dichloromethane was added and stirred at ambient temperature for about 30 hours. The reaction was quenched by aqueous sodium bicarbonate solution. The aqueous layer was extracted with dichloromethane, washed successively with water and then brine, dried over anhydrous sodium sulphate and then the solvent was removed under reduced pressure to yield a crude product. The crude product was crystallized by using ethyl acetate-hexane mixture.

### Step (c) Preparation of Compound of Formula V

Triphosgene (1.5 equiv.) was added to a solution of compound of Formula IV (1 equiv) in dichloromethane at 0° C. with stirring. Pyridine (15 equiv.) was then slowly added. After complete addition, reaction mixture was stirred for about 3 hours at about 0-5° C. and was quenched by drop wise addition of water. The reaction mixture was diluted with dichloromethane, washed with water followed by brine, dried over anhydrous sodium sulphate and concentrated under reduced pressure to yield the title compound.

### Step (d) Preparation of Compound of Formula VI

Tetramethyl guanidine (2.2 equiv.) was added to a solution of compound of Formula V (1 equiv) in dimethylformamide. The reaction mixture was heated at about 90° C. for about 8 hours. The reaction mixture was cooled to an ambient temperature and water was added and extracted with ethyl acetate. The organic layer was washed with water followed by brine, dried over anhydrous sodium sulphate and concentrated under reduced pressure to obtain the desired product.

### Step (e) Preparation of Compound of Formula VII

N-iodosuccinimide (2 equiv.) was added to a solution of compound of Formula VI (1 equiv) in dry acetonitrile:dichloromethane (2:1) at about 0° C. A sodium bisulphite solution was added to the reaction mixture with stirring followed by adding a sodium carbonate solution with further stirring of the reaction mixture. Dichloromethane was evaporated under reduced pressure. The aqueous layer was extracted with ethyl acetate, washed successively with water and then brine, and dried over anhydrous sodium sulphate and then the solvent was removed under reduced pressure to obtain the crude product. The crude product was purified by silica gel column chromatography (thoroughly neutralized with triethyl amine) using 10-20% acetone in hexane to give the product.

### Step (f) Preparation of Compound of Formula VIII

Reagent of Formula R³X (6 equiv.) was added to a solution of compound of Formula VII (1 equiv) and solid sodium bicarbonate (5 equiv.) in acetonitrile. The reaction was carried out in argon atmosphere, at ambient temperature for about 24 hours. The reaction mixture was diluted with ethyl acetate and washed successively with water and then brine, dried over anhydrous sodium sulphate and concentrated under reduced pressure to get the crude product. The crude product was purified by silica gel column chromatography (thoroughly neutralized with triethylamine) using 10-20% acetone in hexane to give the product.

### Step (g) Preparation of Compound of Formula IX

Reagent of Formula R⁵YCOOH (2.5 equiv.), 4-N-dimethylaminopyridine (2.5 equiv) and N,N′-dicyclohexylcarbodiimide (2.5 equiv.) were added to a solution of compound of Formula VIII (1 equiv) in dichloromethane. Pyridine (4 equiv) was added slowly. The reaction mixture was stirred for about 18-24 hours and then filtered through a celite bed. The filtrate was washed successively with water and then brine, dried over anhydrous sodium sulphate and concentrated under reduced pressure to get the crude product. The crude product was purified by silica gel column chromatography (thoroughly neutralized with triethylamine) using 10-20% acetone in hexane.

### Step (h) Preparation of Compound of Formula X

N,N′-carbonyldiimidazole (3 equiv) was added to a solution of compound of Formula IX (1 equiv.) in dimethylformamide: tetrahydrofuran (3:2) at ambient temperature. The reaction mixture was cooled to 0° C., sodium hydride (3 equiv.) was added at once. The reaction mixture was stirred for about 30 minutes. The reaction was quenched by adding ice-cold water and extracted with ethyl acetate. The ethyl acetate layer was washed successively with water and then brine dried over anhydrous sodium sulphate and concentrated under reduced pressure to yield the product.

Step (i) Preparation of compound of Formula XI

The compound of Formula X (1 equiv) and a compound of Formula R—W—NH₂ (3 equiv) were taken in 10% water in acetonitrile and heated at 70-75° C. The reaction mixture was cooled to an ambient temperature and acetonitrile was evaporated under reduced pressure. The resulting residue was taken in ethyl acetate, washed successively with water and then brine, dried over anhydrous sodium sulphate and concentrated under reduced pressure. The resulting residue was purified by column chromatography using 25-30% acetone in hexane to yield the product.

### Step (j) Preparation of Compound of Formula XII

The compound of Formula XI (0.6 mmol) was taken in methanol and refluxed. The reaction mixture was cooled to ambient temperature and methanol was evaporated under reduced pressure. Purification of the crude residue was done over silica gel (thoroughly neutralized by triethyl amine) using 30-35% acetone in hexane or 2-8% methanol in dichloromethane to yield the product.

### Example 3

### Synthetic Procedure of Scheme II

### Step (a) Preparation of Compound of Formula XIII

Ammonia gas was passed through a solution of the compound of Formula X (0.437 mmoles) in dimethylformamide (10 ml) for about 20 minutes at 0° C. The reaction mixture was stirred for about 2 hours. It was extracted with ethyl acetate. The organic layer was washed successively with water and then brine and dried over sodium sulphate to yield the product.

### Step (b) Preparation of Compound of Formula XIV

Compounds of Formula XIV can be prepared by two different methods Method A

The reaction mixture of above step was allowed to stir for about 24 hours at room temperature. The solvent was evaporated and the compound was purified over silica gel using 7% methanol in dichloromethane to yield the product.

### Method B

Potassium-t-butoxide (0.509 mmoles) was added portionwise to a solution of a compound of Formula XIII (0.462 mmoles) in tetrahydrofuran (10 ml) at about 0° C. The reaction mixture was stirred overnight. It was extracted with ethyl acetate, washed successively with water and then brine and dried over sodium sulphate to yield the product.

Step (c) Preparation of compound of Formula XV

A solution of compound of Formula XIV (0.46 mole) in methanol (8 ml) was refluxed. The reaction mixture was cooled to an ambient temperature and methanol was evaporated under reduced pressure to yield the crude product. The crude product was purified over silica gel (thoroughly neutralized by triethyl amine) using 30-35% acetone in hexane or 2-8% methanol in dichloromethane to yield the product.

### Example 4

### Synthetic Procedure of Scheme III

### Step (a) Preparation of Compound of Formula XVIII

Compound of Formula XVI (0.4 mmol) and compound of Formula XVII (1.1 mmol) were taken in dichloromethane and stirred at 25° C. for about 14 hours. Dichloromethane (10 ml) was evaporated under reduced pressure and the resulting residue was purified by column chromatography using hexane: acetone: triethyl amine as the eluant to yield the product.

### Step (b) Preparation of Compound of Formula XIX

Compound of Formula XVIII (0.2 mmol) was taken in methanol (5 ml) and refluxed for about 18 hours. The reaction mixture was cooled to 30° C., the solvent was evaporated under reduced pressure and the resulting residue was purified by column chromatography over 100-200-mesh silica get using dichloromethane-methanol as eluant to yield the product.

The following illustrative compounds were analogously prepared by following the above general procedures.

Compound No. 1: 11,12-dideoxy-3-O-decladinosyl-3-O— (2-pyridyl acetyl)-5-O— (3′-N-desmethyl-3′-N-ethyl)-6-O— methyl-12,11-[oxycarbonyl-((4-(benzoimidazol-1-yl)-pentyl)-imino)]erythromycin A; Mass: 934.41,

Compound No. 2: 11,12-dideoxy-3-O-decladinosyl-3-O— (2-pyridyl acetyl)-5-O— (3′-N-desmethyl-3′-N-allyl)-6-O— methyl-12,11-[oxycarbonyl-((4-(benzoimidazol-1-yl)-pentyl)-imino)]erythromycin A; Mass: 946.36,

Compound No. 3: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(1H-imidazo[4,5-b]pyridin-1-yl)-pentyl)-imino)]erythromycin A; Mass: 947.49,

Compound No. 4: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(3H-imidazo[4,5-b]pyridin-3-yl)-pentyl)-imino)]erythromycin A; Mass: 947.35,

Compound No. 5: 11,12-dideoxy-3-O-decladinosyl-3-O— (2-pyridyl acetyl)-5-O— (3′-N-desmethyl-3′-N-allyl)-6-O-methyl-12,11-[oxy carbonyl-(3-(4-pyridin-3-yl-phenoxy)-imino)]erythromycin A; Mass: 972.48,

Compound No. 6: 11,12-dideoxy-3-O-decladinosyl-3-O— (2-pyridyl acetyl)-5-O— (3′-N-desmethyl-3′-N-allyl)-6-O-methyl-12,11-[oxycarbonyl-((1-(9-(4-amino-butyl)-9H-purin-6-yl)-3-butyl)-urea)-imino)]erythromycin A; Mass: 1048.67,

Compound No. 7: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((1-(9-(4-amino-butyl)-9H-purin-6-yl)-3-(2,6-difluoro-phenyl)-urea)-imino)]erythromycin A; Mass: 1104.63,

Compound No. 8: 11,12-dideoxy-3-O-decladinosyl-3-O— (2-pyridyl acetyl)-5-O— (3′-N-desmethyl-3′-N-allyl)-6-O-methyl-12,11-[oxycarbonyl-((1-allyl-3-(9-(4-amino-butyl)-9H-purin-6-yl)-urea)-imino)]erythromycin A; Mass: 1032.40,

Compound No. 9: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((1-(9-(4-amino-butyl)-9H-purin-6-yl)-3-(4-fluoro-phenyl)-urea)-imino)]erythromycin A; Mass: 1086.32,

Compound No. 10: 11,12-dideoxy-3-O-decladinosyl-3-O— (2-pyridyl acetyl)-5-O— (3′-N-desmethyl-3′-N-allyl)-6-O-methyl-12,11-[oxycarbonyl-((3-(3-pyridin-3-yl-phenoxy)-propyl)-imino)]erythromycin A; Mass: 971.26,

Compound No. 11: 11,12-dideoxy-3-O-decladinosyl-3-O-(2-pyridyl acetyl)-5-O-(3′-N-desmethyl-3′-N-allyl)-6-O-methyl-12,11-[oxycarbonyl-imino)]erythromycin A; Mass: 760.49,

Compound No. 12: 11,12-dideoxy-3-O-decladinosyl-3-O-(3-pyridyl acetyl)-5-O-(3′-N-desmethyl-3′-N-allyl)-6-O-methyl-12,11-[oxycarbonyl-imino)]erythromycin A; Mass: 760.56, and

Compound No. 13: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(3-thiophen-3-yl-phenoxy)-propyl)-imino)]erythromycin A; Mass: 976.67.

### Pharmacological Activity

Compounds disclosed herein displayed antibacterial activity in vitro especially against strains which are resistant to macrolides either due to efflux (mef strains) or ribosomal modification (erm) strains. These compounds are useful in the treatment of community acquired pneumonia, upper and lower respiratory tract infections, skin and soft tissue infections, hospital acquired lung infections, bone and joint infections, and other bacterial infections, for example, mastitis, catheter infection, foreign body, prosthesis infections or peptic ulcer disease.

Minimum inhibitory concentration (MIC) has been an indicator of in vitro antibacterial activity widely used in the art.

### Medium—

a) Cation adjusted Mueller Hinton Agar (MHA-Difco)

### b) Trypticase Soya Agar (TSA)

Inoculum preparation—The cultures were streaked on TSA for aerobic cultures and MHA with 5% sheep blood for fastidious cultures. Aerobic cultures were incubated at 37° C. for about 18-24 hours. Fastidious cultures were incubated CO₂ incubation (5% CO₂) at 37° C. for about 18-24 hours. Three to four well isolated colonies were taken and saline suspension were prepared in sterile densimat tubes. The turbidity of the culture was adjusted to 0.5-0.7 Mc Farland standard (1.5×10⁸ CFU/ml (Colony Forming Unit)/ml). The cultures were diluted 10-fold in saline to get inoculum size of approximately 1−2×10⁷ organisms/ml.

Preparation of drug concentration—1 mg/ml concentration of stock solution of drugs was prepared in dimethyl sulphoxide/distilled water/solvent given in National Committee for Clinical Laboratory Standards (NCCLS) manual. Serial two-fold dilutions of the compounds and standard drugs were prepared as per NCCLS manual. Stock solution can be changed according to the need of the experiment.

Preparation of Agar Plates—Two ml of respective drug concentration was added to 18 ml of Molten Mueller Hinton agar to get the required range, for example 0.015 μg/ml-16 μg/ml. For fastidious culture added 1 ml of sheep blood in Molten Mueller Hinton agar.

For control MHA and MHA with 5% sheep blood plates without antibiotic for each set were prepared. One MHA and MHA with 5% sheep blood plates without antibiotic for determining quality check for media was prepared.

Preparation of Teflon template—1 μg of each culture on each plate was replicated with the help of replicator (Denley's multipoint replicator). The spots were allowed to dry and the plates were incubated for about 18-24 hours at 37° C. Fastidious cultures were incubated at 37° C. in CO₂ incubator. The results were noted with the control plates.

Endpoint definition—The concentration of drug at which there was complete disappearance of growth spot or formation of less than 10 colonies per spot was considered as Minimum Inhibitory Concentration (MIC).

The MICs of quality control (QC) strains were plotted on the QC chart for agar dilution method. If the MICs were within the range, the results interpreted by comparing MICs of standards against all organisms with those of test compounds.

### Precautions & Quality Control Measures

### Quality Control Strains

Staphylococcus aureus ATCC 29213
Enterococcus faecalis ATCC 29212
Eschericia coli ATCC 25922
Pseudomonas aeruginosa ATCC 27853
All 60 cultures were visually checked for purity.

Media Control: Performed NCCLS disc diffusion assay using 10 μg discs of Gentamicin (Difco) against Ps. aeruginosa ATCC 27853. A zone diameter of 16-21 mm should be considered for optimum cation (Magnesium and Calcium) content of the media. Plotted the diameter in the media QC chart.

### REFERENCES

National Committee for Clinical Laboratory Standards (NCCLS), Methods for Dilution Antimicrobial Susceptibility Tests for Bacteria That Grow Aerobically—Fifth Edition; Approved Standard. M7-A5, Vol. 20. No. 2 (January 2000).
National Committee for Clinical Laboratory Standards, Performance Standards for Antimicrobial Susceptibility Testing—Twelfth informational supplement, M 100-S12, Vol. 22 No. 1 (January 2002).

Results: compounds 1, 2, 5-9, and 11 have been examined and have shown activity against Staphylococcus aureus strains 25923, Streptococcus pneumoniae strains 6303, 49619, 3579, 3390, 4745, 994, 5055, and 5051, Haemophilus influenzae strains 49247, and 38, Moraxella catarrhalis strains 8176 and M6, Streptococcus pyogenes strains 1721 erm B and 2534 erm B, Enterococcifaecalis strains 29212 and 346, and Enterococcifaecium strain 6A.
a) The compounds disclosed herein exhibited MIC values against Staphylococcus aureus in the range between about 0.125 zg/mL to about 16 μg/mL, for example from about 0.125 μg/mL to about 4 μg/mL.
b) The compounds disclosed herein exhibited MIC values against sensitive Streptococcus pneumoniae in the range between about 0.015 μg/mL to about 0.5 μg/mL.
c) The compounds disclosed herein exhibited MIC values against resistant Streptococcus pneumoniae in the range between about 0.06 μg/mL to about 8 μg/mL.
d) The compounds disclosed herein exhibited MIC values against Haemophilus influenzae in the range between about 0.06 μg/mL to about 4 μg/mL.
e) The compounds disclosed herein exhibited MIC values against Moraxella species in the range between about 0.25 μg/mL to about 8 μg/mL.
f) The compounds disclosed herein exhibited MIC values against telithromycin resistant Streptococcus pneumoniae in the range between about 1 μg/mL to about >16 μg/mL.
g) The compounds disclosed herein exhibited MIC values against sensitive Streptococcus pyogenes in the range between about 0.015 μg/mL to about 0.25 μg/mL.
h) The compounds disclosed herein exhibited MIC values against resistant Streptococcus pyogenes in the range between about 0.06 μg/mL to about >16 μg/mL.
i) The compounds disclosed herein exhibited MIC values against MRSA about >16 μg/mL.
j) The compounds disclosed herein exhibited MIC values against sensitive Enterococci species in the range between about 0.125 μg/mL.
k) The compounds disclosed herein exhibited MIC values against resistant Enterococci species in the range between about 0.5 μg/mL to about >16 μg/mL.

We claim:

1. Compounds having the structure of Formula I, 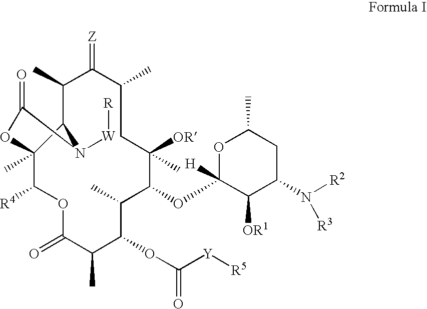
pharmaceutically acceptable salts, pharmaceutically acceptable solvates, stereoisomers, tautomers, racemates, prodrugs, metabolites and polymorphs thereof, wherein:
R¹ is hydrogen or a hydroxyl protecting group;
R² and R³ are independently hydrogen, alkyl, alkenyl, alkynyl, cycloalkyl, aryl, heterocycle, aralkyl or (heterocycle)alkyl, with the proviso that R² and R³ are not simultaneously methyl;
R⁴ is hydrogen, alkyl, alkenyl or alkynyl;
R⁵ is aryl, heterocycle or alkyl;
R is no atom, aryl or heterocycle;
R′ is alkyl or —(CH₂)_{q}—U—V, (wherein q is an integer of from 1 to 4. U is alkenyl or alkynyl, and V is hydrogen, aryl or heterocycle);
W is hydrogen or —(CH₂)ₘ-{wherein m is an integer of from 2 to 6, CH₂ of (CH₂)ₘ group is optionally interrupted by O, S or NR⁶, (wherein R⁶ can be hydrogen, alkyl, cycloalkyl, alkenyl, heterocyclyl, (heterocyclyl)alkyl, alkynyl, aryl or aralkyl), and one of the hydrogen atom of (CH₂)ₘ is optionally replaced by alkyl, hydroxy or alkoxy};
Y is -Q(CH₂)ₖ—, (wherein k is an integer of from 1 to 6, Q is no atom, O or NR⁷, wherein R⁷ can be hydrogen or alkyl, and one of the hydrogen atoms of (CH₂)ₖ is optionally replaced by alkyl, hydroxy or alkoxy); and
Z is O, S or NOR⁸, (wherein R⁸ can be hydrogen, alkyl or aralkyl).

2. Compounds of claim 1, wherein R is no atom or heterocycle optionally substituted with —NHCONHR⁹, wherein R⁹ is alkyl, alkenyl, alkynyl, cycloalkyl, aryl, aralkyl, (heterocycle) alkyl or heterocyclyl.

3. Compounds of claim 1, wherein R is benziimidazol-1-yl, (1H-imidazo[4,5-b]pyridin-1-yl, (3H-imidazo[4,5-b]pyridin-3-yl, (4-pyridin-3-yl)-phenyl, 1-butyl-3-(9H-purin-6-yl) urea, 1-(2,6-difluoro-phenyl)-3-(9H-purin-6-yl) urea, 1-allyl-3-(9H-purin-6-yl)-urea, 1-(4-fluoro-phenyl)-3-(9H-purin-6-yl urea, (3-pyridin-3-yl)-phenyl and (3-thiophen-3-yl)-phenyl.

4. Compounds of claim 1, wherein W is hydrogen or —(CH₂)ₘ—, wherein (CH₂)ₘ is optionally interrupted by O, S or NR⁶, where R⁶ is hydrogen, alkyl, cycloalkyl, alkenyl, heterocyclyl, (heterocyclyl)alkyl, alkynyl, aryl or aralkyl, and m is an integer of from 2 to 6.

5. Compounds of claim 1, wherein R² is methyl, and R³ is alkyl or alkenyl, with the proviso that R³ is not methyl.

6. Compounds of claim 1, wherein R⁵ is monocyclic heterocycle having N as heteroatom(s).

7. Compounds of claim 1, wherein R¹ is hydrogen; Z is oxygen; R⁴ is alkyl; and R¹ is alkyl.

8. A compound which is:
11,12-dideoxy-3-O-decladinosyl-3-O-(2-pyridylacetyl)-5-O-(3′-N-desmethyl-3′-N-ethyl)-6-O-methyl-12,11-[oxycarbonyl-((4-(benzoimidazol-1-yl)-pentyl)-imino)]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(benzoimidazol-1-yl)-pentyl)-imino)]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(1H-imidazo[4,5-b]pyridin-1-yl)-pentyl)-imino)]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(3H-imidazo[4,5-b]pyridin-3-yl)-pentyl)-imino)]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-(3-(4-pyridin-3-yl-phenoxy)-imino)]erythromycin A,
11,12-dideoxy-3-O-decladinosyl-3-O— (2-pyridylacetyl)-5-O— (3′-N-desmethyl-3′-N-allyl)-6-O-methyl-12,11-[oxycarbonyl-((1-(9-(4-amino-butyl)-9H-purin-6-yl)-3-butyl)-urea)-imino)]erythromycin A,
11,12-dideoxy-3-O-decladinosyl-3-O— (2-pyridylacetyl)-5-O— (3′-N-desmethyl-3′-N-allyl)-6-O-methyl-12,11-[oxycarbonyl-((1-(9-(4-amino-butyl)-9H-purin-6-yl)-3-(2,6-difluoro-phenyl)-urea)-imino)]erythromycin A,
11,12-dideoxy-3-O-decladinosyl-3-O— (2-pyridylacetyl)-5-O— (3′-N-desmethyl-3′-N-allyl)-6-O-methyl-12,11-[oxycarbonyl-((1-allyl-3-(9-(4-amino-butyl)-9H-purin-6-yl)-urea)-imino)]erythromycin A,
11,12-dideoxy-3-O-decladinosyl-3-O— (3-pyridylacetyl)-5-O— (3′-N-desmethyl-3′-N-allyl)-6-O-methyl-12,11-[oxycarbonyl-((1-(9-(4-amino-butyl)-9H-purin-6-yl)-3-(4-fluoro-phenyl)-urea)-imino)]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((3-(3-pyridin-3-yl-phenoxy)-propyl)-imino)]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-imino)]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-imino)]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((3-(3-thiophen-3-yl-phenoxy)-propyl)-imino)]erythromycin A, and
pharmaceutically acceptable salts, pharmaceutically acceptable solvates, stereoisomers, tautomers, racemates, prodrugs, metabolites and polymorphs thereof.

9. A pharmaceutical composition comprising therapeutically effective amounts of one or more compounds of claim 1 together with one or more pharmaceutically acceptable carriers, excipients or diluents.

10. A method for treating or preventing the condition caused by or contributed to by bacterial infection comprising administering to a mammal in need thereof therapeutically effective amounts of one or more compound of claim 1 or one or more pharmaceutical compositions of claim 9.

11. The method of claim 10, wherein the condition is selected from community acquired pneumonia, upper and lower respiratory tract infections, skin and soft tissue infections, hospital acquired lung infections or bone and joint infections, mastitis, catether infection, foreign body or prosthesis infections.

12. The method of claim 10, wherein the bacterial infection is caused by Gram-positive, Gram-negative or anaerobic bacteria.

13. The method of claim 11, wherein Gram-positive, Gram-negative or anaerobic bacteria. is selected from Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus and Enterobactericeae.

14. The method of claim 12, wherein the bacterium is a cocci bacterium.

15. The method of claim 13, wherein the bacterium is drug resistant.

16. A method for preparing compounds of Formula XII, 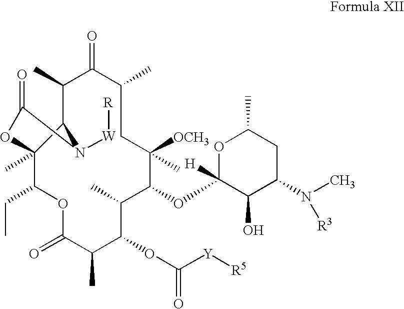
pharmaceutically acceptable salts, pharmaceutically acceptable solvates, stereoisomers, tautomers, racemates, prodrugs, metabolites or polymorphs thereof, wherein:
R³ is hydrogen, alkyl, alkenyl, alkynyl, cycloalkyl, aryl, heterocycle, aralkyl or (heterocycle)alkyl, with the proviso that R³ is not methyl;
R⁵ is aryl, heterocycle or alkyl;
R is no atom, aryl or heterocycle;
W is hydrogen or —(CH₂)ₘ-{wherein m is an integer of from 2 to 6, CH₂ of (CH₂)ₘ group is optionally interrupted by O, S or NR⁶, (wherein R⁶ can be hydrogen, alkyl, cycloalkyl, alkenyl, heterocyclyl, (heterocyclyl)alkyl, alkynyl, aryl or aralkyl), and one of the hydrogen atom of (CH₂)ₘ is optionally replaced by alkyl, hydroxy or alkoxy}; and
Y is -Q(CH₂)ₖ—, (wherein k is an integer of from 1 to 6, Q is no atom, O or NR⁷, wherein R⁷ can be hydrogen or alkyl, and one of the hydrogen atoms of (CH₂)ₖ is optionally replaced by alkyl, hydroxy or alkoxy)
the method comprising:
(a) hydrolyzing clarithromycin of Formula II, 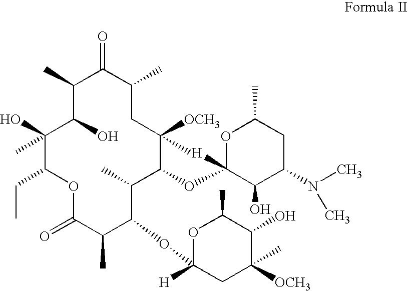
to form a compound of Formula III, 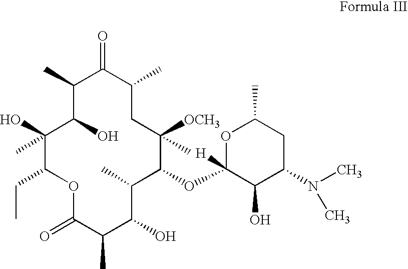
(b) protecting the compound of Formula III with one or more reagents of Formula R¹₂O or R¹X (wherein X is halogen) to form a compound of Formula IV, 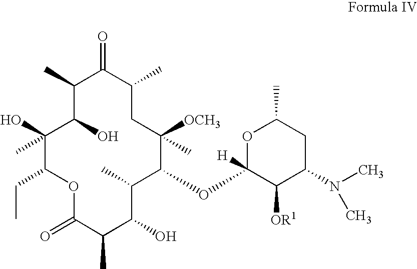
(c) reacting the compound of Formula IV with one or more reagents to form a compound of Formula V, 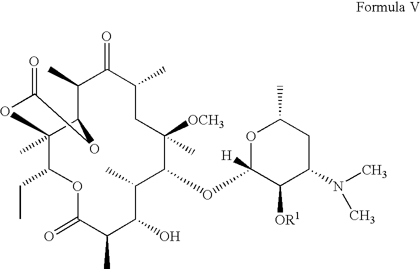
(d) reacting the compound of Formula V with one or more organic bases to form a compound of Formula VI, 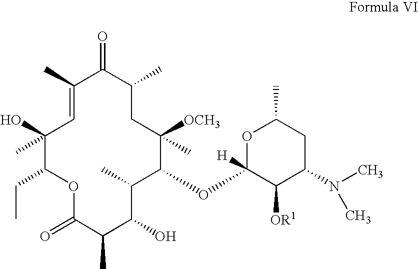
(e) desmethylating the compound of Formula VI at 3′-N-dimethyl group to form a compound of Formula VII, 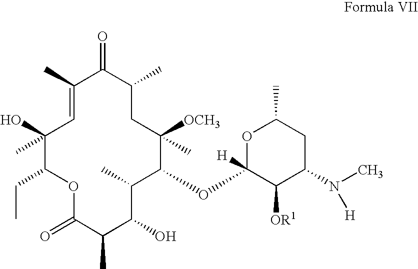
(f) alkylating the compound of Formula VII with one or more reagents of Formula R³CHO, R³₂CO or R³X to form a compound of Formula VIII (wherein R³ is the same as defined earlier), 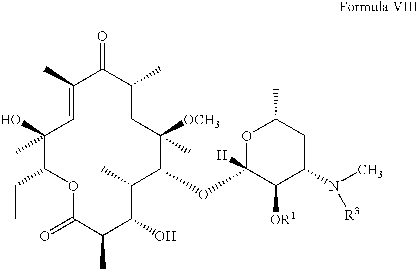
(g) acylating the compound of Formula VIII with one or more reagents of Formula R⁵YCOOH, (R⁵YCO)₂O, R⁵YCOX or R⁵YCOOR¹⁰ (wherein R¹⁰ is a leaving group selected from pivaloyl, p-toleuensulfonyl, isobutoxycarbonyl, ethoxycarbonyl and isopropoxycarbonyl) to form a compound of Formula IX (wherein Y and R⁵ are the same as defined earlier), 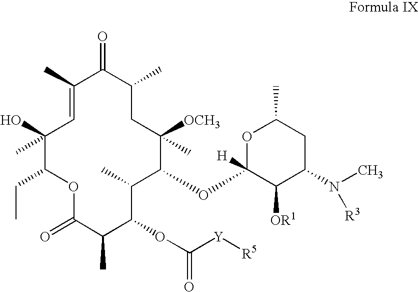
(h) reacting the compound of Formula IX with N,N′-carbonyl diimidazole to form a compound of Formula X, 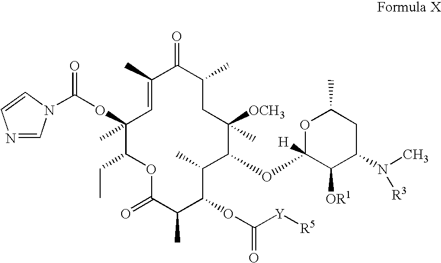
(i) reacting the compound of Formula X with a compound of Formula R—W—NH₂ to form a compound of Formula XI (wherein R and W are the same as defined earlier), 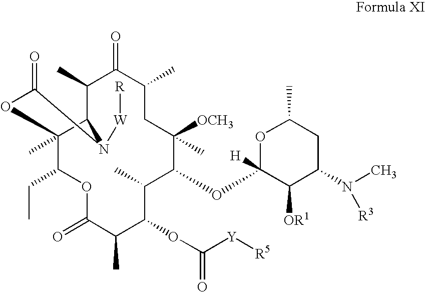
(j) deprotecting the compound of Formula XI to form a compound of Formula XII.

17. A method for preparing compounds of Formula XV, 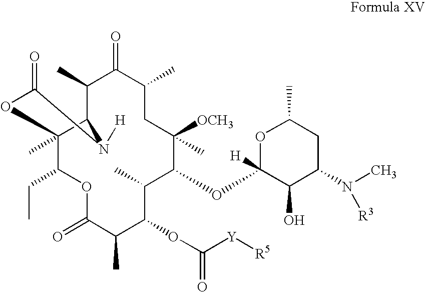
pharmaceutically acceptable salts, pharmaceutically acceptable solvates, stereoisomers, tautomers, racemates, prodrugs, metabolites or polymorphs thereof, wherein:
R³ is hydrogen, alkyl, alkenyl, alkynyl, cycloalkyl, aryl, heterocycle, aralkyl or 14 (heterocycle)alkyl, with the provisio that R³ is not methyl;
R⁵ is aryl, heterocycle or alkyl;
Y is -Q(CH₂)ₖ—, (wherein k is an integer of from 1 to 6, Q is no atom, O or NR⁷, wherein R⁷ can be hydrogen or alkyl, and one of the hydrogen atoms of (CH₂)ₖ is optionally replaced by alkyl, hydroxy or alkoxy);
the method comprising:
(a) reacting the compound of Formula X, 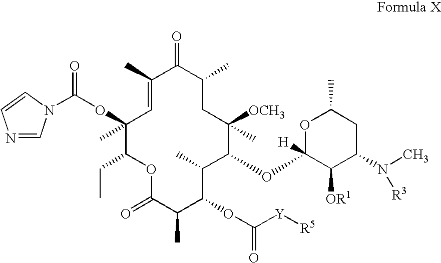
with ammonia to form a compound of Formula XIII (wherein R³, R¹, Y and R⁵ are the same as defined earlier), 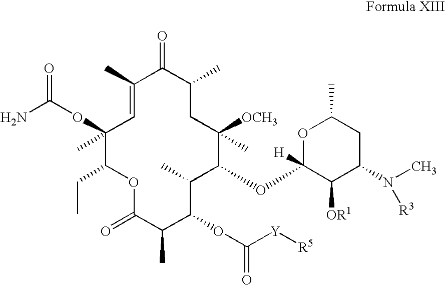
(b) cyclizing the compound of Formula XIII to form a compound of Formula XIV, 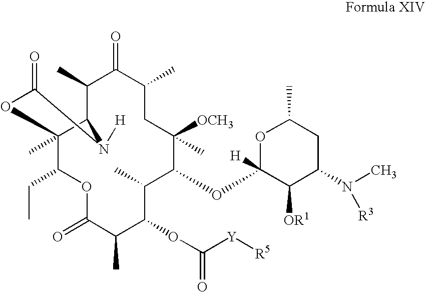
(c) deprotecting the compound of Formula XIV to form a compound of Formula XV.

18. A process for preparing compounds of Formula XIX, 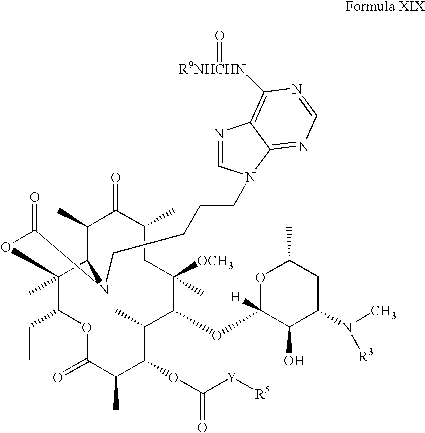
pharmaceutically acceptable salts, pharmaceutically acceptable solvates, stereoisomers, tautomers, racemates, prodrugs, metabolites or polymorphs thereof wherein:
R³ is hydrogen, alkyl, alkenyl, alkynyl, cycloalkyl, aryl, heterocycle, aralkyl or (heterocycle)alkyl, with the provisio that R³ is not methyl;
R⁵ is aryl, heterocycle or alkyl;
Y is -Q(CH₂)ₖ—, (wherein k is an integer of from 1 to 6, Q is no atom, O or NR⁷, wherein R⁷ can be hydrogen or alkyl, and one of the hydrogen atoms of (CH₂)ₖ is optionally replaced by alkyl, hydroxy or alkoxy);
R⁹ is alkyl, alkenyl, alkynyl, cycloalkyl, aryl, aralkyl, (heterocycle)alkyl or heterocyclyl;
the method comprising:
(a) reacting the compound of Formula XVI with a compound of Formula XVII, 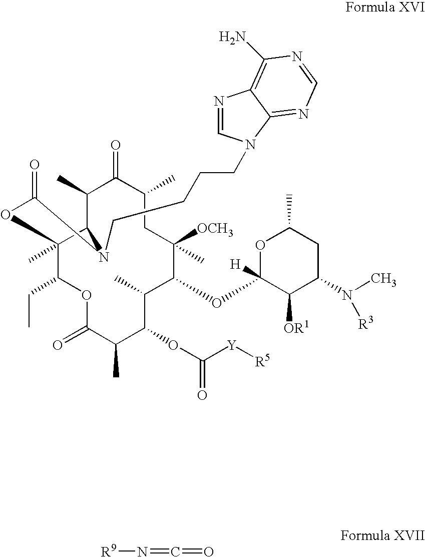
to form a compound of Formula XVIII (wherein R¹, R⁹, R³, Y and R⁵ are the same as defined earlier), and 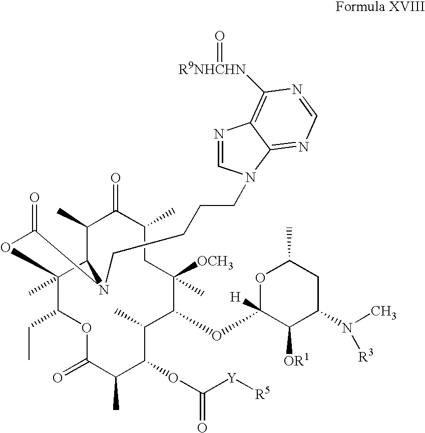
(b) deprotecting the compound of Formula XVIII to form a compound of Formula XIX.
